(19) United States
(12) Patent Application Publication
(10) Pub. No.: US 2015/0031032 A1
(43) Pub. Date: Jan. 29, 2015

(54) Methods and Compositions for Determining Indication for Prostate Biopsy

(71) Applicant: Wake Forest University Health Services, Winston-Salem, NC (US)

(72) Inventor: Jianfeng Xu, Clemmons, NC (US)

(21) Appl. No.: 14/444,945

(22) Filed: Jul. 28, 2014

Related U.S. Application Data
(60) Provisional application No. 61/859,154, filed on Jul. 26, 2013.

(51) Int. Cl. C12Q 1/68; G06F 19/00

(52) U.S. Cl. CPC C12Q 1/6886; G06F 19/3431; C12Q 2600/156
USPC 435/6.11; 702/19

(57) ABSTRACT

The present invention provides a method of identifying a subject for whom a prostate biopsy is indicated, comprising: a) determining, from a nucleic acid sample obtained from the subject, a genotype for the subject at a plurality of biallelic polymorphic loci, wherein each of said plurality has an associated allele and an unassociated allele, wherein the genotype is selected from the group consisting of homozygous for the associated allele, heterozygous, and homozygous for the unassociated allele; b) calculating a genetic risk score (GRS) for the subject based on the genotype determined in step (a); and c) analyzing the GRS of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated.

### STATEMENT OF PRIORITY

This application claims the benefit, under 35 U.S.C. §119(e), of U.S. Provisional Application Ser. No. 61/859,154, filed Jul. 26, 2013, the entire contents of which are incorporated by reference herein.

### STATEMENT OF GOVERNMENT SUPPORT

This invention was made with government support under Grant No. CA148463 awarded by the National Institutes of Health. The United States Government has certain rights in this invention.

### FIELD OF THE INVENTION

The present invention provides methods and compositions directed to assessing a subject's risk of developing prostate cancer and to determining whether a prostate biopsy is indicated for a subject by analyzing multiple single nucleotide polymorphisms in nucleic acid of a subject in combination with additional biomarkers, such as, e.g., prostate specific antigen (PSA) level or prostate health index (PHI).

### BACKGROUND OF THE INVENTION

Prostate cancer (PCa) is the most common solid organ malignancy affecting American men and the second leading cause of cancer related death. Approximately one million prostate biopsies are performed yearly in the U.S. The vast majority of these biopsies are performed due to elevated levels of the PCa marker prostate-specific antigen (PSA). However, only a quarter of these biopsies result in a diagnosis of PCa, highlighting the inadequate performance of currently available parameters such as PSA to predict PCa. Persistently elevated PSA levels and/or other clinical parameters that prompted initial biopsies contribute to stress and anxiety among both patients and their urologists. Thus, the predictive performance of currently available clinical parameters such as PSA is limited. Furthermore, management of men following negative prostate biopsy for prostate cancer is challenging. A more relevant approach employing biomarkers is urgently needed to better determine the need for initial and repeat prostate biopsy and assess an individual's risk.

Single nucleotide polymorphisms (SNPs) are stable genetic markers throughout the human genome, which can be tested for their association with various disease traits. These markers can be tested at birth and will not change in a patient's lifetime and thus represent a relevant form of biomarkers that predict lifetime risk to disease as opposed to an immediate risk.

Numerous PCa risk-associated single nucleotide polymorphisms (SNPs) have been discovered from genome-wide association studies (GWAS). In particular, 33 SNPs have been consistently found, in several populations of Caucasian race, to be associated with prostate cancer (PCa) risk (Table 1). These risk-associated SNPs have been consistently replicated in multiple case-control study populations of European descent. Although each of these SNPs is only moderately associated with PCa risk, a genetic score based on a combination of risk-associated SNPs can be used to more particularly identify a subject's risk of developing PCa. These risk-associated SNPs have broad practical applications because they are common in the general population.

The present invention overcomes previous shortcomings in the art by identifying a subject's risk of developing prostate cancer and identifying subjects for whom a prostate biopsy is indicated.

### SUMMARY OF THE INVENTION

The present invention provides a method of identifying a subject for whom a prostate biopsy is indicated, comprising: a) determining, from a nucleic acid sample obtained from the subject, a genotype for the subject at a plurality of biallelic polymorphic loci, wherein each of said plurality has an associated allele and an unassociated allele, wherein the genotype is selected from the group consisting of homozygous for the associated allele, heterozygous, and homozygous for the unassociated allele; b) calculating a genetic risk score (GRS) for the subject based on the genotype determined in step (a); and c) analyzing the GRS of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated. The method of this invention can further comprise the step of d) performing a prostate biopsy on the subject identified as a subject for whom a prostate biopsy is indicated according to step (c).

In addition, the present invention provides a method of determining whether to perform a prostate biopsy on a subject, comprising: a) determining, from a nucleic acid sample obtained from the subject, a genotype for the subject at a plurality of biallelic polymorphic loci, wherein each of said plurality has an associated allele and an unassociated allele, wherein the genotype is selected from the group consisting of homozygous for the associated allele, heterozygous, and homozygous for the unassociated allele; b) calculating a genetic risk score (GRS) for the subject based on the genotype determined in step (a); c) analyzing the GRS of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated; d) performing a prostate biopsy on the subject if the subject is identified as a subject for whom a prostate biopsy is indicated according to step (c); and e) not performing a prostate biopsy on the subject if the subject is not identified as a subject for whom a prostate biopsy is indicated according to step (c).

Further provided herein is a method of identifying a subject for whom a prostate biopsy is indicated, comprising: a) determining, from a nucleic acid sample obtained from the subject, a genotype for the subject at a plurality of biallelic polymorphic loci, wherein each of said plurality has an associated allele and an unassociated allele, wherein the genotype is selected from the group consisting of homozygous for the associated allele, heterozygous, and homozygous for the unassociated allele; b) calculating a genetic risk score (GRS) for the subject based on the genotype determined in step (a); c) determining, from a sample obtained from the subject, a p2PSA level, a free PSA (fPSA) level and a total PSA (tPSA) level for the subject; d) calculating a prostate health index (PHI) for the subject based on the p2PSA level, fPSA level and tPSA level determined in step (c); c) analyzing the GRS and PHI of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated; and d) performing a prostate biopsy on the subject identified as a subject for whom a prostate biopsy is indicated according to step (c).

The present invention also provides a method of determining whether to perform a prostate biopsy on a subject, comprising: a) determining, from a nucleic acid sample obtained from the subject, a genotype for the subject at a plurality of biallelic polymorphic loci, wherein each of said plurality has an associated allele and an unassociated allele, wherein the genotype is selected from the group consisting of homozygous for the associated allele, heterozygous, and homozygous for the unassociated allele; b) calculating a genetic risk score (GRS) for the subject based on the genotype determined in step (a); c) determining, from a sample obtained from the subject, a p2PSA level, a free PSA (fPSA) level and a total PSA (tPSA) level for the subject; d) calculating a prostate health index (PHI) for the subject based on the p2PSA level, fPSA level and tPSA level determined in step (c); c) analyzing the GRS and PHI of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated; d) performing a prostate biopsy on the subject if the subject is identified as a subject for whom a prostate biopsy is indicated according to step (c); and e) not performing a prostate biopsy on the subject if the subject is not identified as a subject for whom a prostate biopsy is indicated according to step (c).

Also provided herein is a method of identifying a subject for whom a prostate biopsy is indicated, comprising: a) determining, from a sample obtained from the subject, a p2PSA level, a free PSA (fPSA) level and a total PSA (tPSA) level for the subject; b) calculating a prostate health index (PHI) for the subject based on the p2PSA level, fPSA level and tPSA level determined in step (a);

c) analyzing the PHI of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated; and

d) performing a prostate biopsy on the subject identified as a subject for whom a prostate biopsy is indicated according to step (c).

Furthermore, the present invention provides a method of determining whether to perform a prostate biopsy on a subject, comprising: a) determining, from a sample obtained from the subject, a p2PSA level, a free PSA (fPSA) level and a total PSA (tPSA) level for the subject; b) calculating a prostate health index (PHI) for the subject based on the p2PSA level, fPSA level and tPSA level determined in step (a); c) analyzing the PHI of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated; d) performing a prostate biopsy on the subject if the subject is identified as a subject for whom a prostate biopsy is indicated according to step (c); and e) not performing a prostate biopsy on the subject if the subject is not identified as a subject for whom a prostate biopsy is indicated according to step (c).

In additional embodiments, the present invention provides a chart for determining prostate cancer detection rate as a percentile value, said chart comprising: a) a first region comprising a first, second and third prostate specific antigen (PSA) value; b) a second region, adjacent to each respective first, second and third PSA value of the first region, comprising a first, second and third genetic risk score (GRS) value and an average detection rate value (ALL), wherein the first, second and third GRS values are the same for each of the respective PSA values to which the first, second and third GRS values are adjacent; c) a third region, adjacent to the second region, comprising a reference bar to show a prostate cancer detection rate in percentiles ranging from 1% to 100%; and d) a fourth region comprising a grid, aligned below the third region and in parallel with the first, second and third GRS values and the ALL value of the second region for each of the respective first, second and third PSA values of the first region, showing a percentile value that specifies a prostate cancer detection rate for each of the first, second and third GRS values and the ALL value in the second region for each of the first, second and third PSA values in the first region.

### BRIEF DESCRIPTION OF THE DRAWINGS

FIGS. 1a-f. Distribution of estimated risk for each of the three models. These models consist of genetic score (GS), GS plus three pre-biopsy variables (GS+3 variables), and GS plus three pre-biopsy and 3 post-biopsy variables (GS+5 variables). FIGS. 1d, e and f show that, for each respective model (GS, GS+3, GS+5), the PCa detection rate trends upward in reflection of increasing risk quartile.

FIGS. 2a-b. Detection rates for prostate cancer for men below or above the median estimated risk based on a) the genetic model (genetic score of 33 PCa risk-associated SNPs) and b) the best clinical model (with five parameters: age, family history, free/total PSA ratio, prostate volume, and number of cores at initial biopsy). Detection rates for the genetic model were directly estimated. Detection rates for the best clinical model were estimated based on four-fold cross validation. Vertical lines in each bar represent 95% CI of detection rates.

FIG. 3. Detection rates for prostate cancer for men below or above the median estimated risk based on the best clinical model (age, family history, free/total PSA ratio, prostate volume, and number of cores at initial biopsy), and stratified by genetic risk (lower or higher half of genetic risk). Vertical lines in each bar represent 95% CI of detection rates.

FIGS. 4a-c. Detection rates for high-grade prostate cancer for men below or above the median estimated risk based on a) the genetic model, b) the best clinical model (age, family history, free/total PSA ratio, prostate volume, and number of cores at initial biopsy), and c) the best clinical model and stratified by genetic risk (lower or higher half of genetic risk). Vertical lines in each bar represent 95% CI of detection rates.

FIGS. 5a-f. Detection rate of PCa and high grade PCa among men with various estimated PCa risk based on genetic score, clinical variables and combination of both.

FIGS. 6a-b. Detection rate of PCa and high-grade PCa among men with various estimated PCa risk based on the best clinical variables, stratified by genetic risk.

FIG. 7. The Xu's chart for prostate biopsy (PSA+GRS) (Caucasian). The average detection rates of prostate cancer (PCa) from biopsy (circles) and 95% confidence intervals (black horizontal lines) are plotted for patients at different prostate-specific antigen (PSA) levels. In addition, within each PSA level group, the average PCa cancer detection rates and 95% confidence intervals are plotted for individuals in the with low genetic risk score (GRS) (<0.5, triangle), intermediate-GRS (0.5-1.5, square) and high GRS (>1.5, diamond). Data were based on a total of 4499 biopsy patients from a population-based biopsy cohort from Sweden and the placebo arm of the REDUCE (reduction by dutasteride of prostate cancer events) trial described herein. The percentage of patients with low, intermediate and high GRS in each PSA level group is described in parentheses.

FIG. 8. The Xu's chart for prostate biopsy (PSA+GRS) (Chinese). The average detection rates of prostate cancer (PCa) from biopsy (circles) and 95% confidence intervals (black horizontal lines) are plotted for patients at different prostate-specific antigen (PSA) levels. In addition, within each PSA level group, the average PCa cancer detection rates and 95% confidence intervals are plotted for individuals with low genetic risk score (GRS) (<0.5, triangle), intermediate-GRS (0.5-1.5, square) and high GRS (>1.5, diamond). Data were based on a total of 630 biopsy patients from two tertiary hospitals in Shanghai, China. The percentage of patients with low, intermediate and high GRS in each PSA level group is described in parentheses.

FIG. 9. The Xu's chart for prostate biopsy (PSA+PHI) (Chinese). The average detection rates of prostate cancer (PCa) from biopsy (circles) and 95% confidence intervals (black horizontal lines) are plotted for patients at different prostate-specific antigen (PSA) levels. In addition, within each PSA level group, the average PCa cancer detection rates and 95% confidence intervals are plotted for individuals in the lowest quartile (Q1), intermediate quartiles (Q2-Q3) and highest quartile (Q4) for prostate health index (PHI). Data were based on a total of 630 biopsy patients from two tertiary hospitals in Shanghai, China.

FIG. 10. The Xu's chart for prostate biopsy [PSA+(GRS+PHI) (Chinese). The average detection rates of prostate cancer (PCa) from biopsy (circles) and 95% confidence intervals (black horizontal lines) are plotted for patients at different prostate-specific antigen (PSA) levels. In addition, within each PSA level group, the average PCa cancer detection rates and 95% confidence intervals are plotted for individuals in the lowest quartile (Q1), intermediate quartiles (Q2-Q3) and highest quartile (Q4) for PHI and GRS. Data were based on a total of 630 biopsy patients from two tertiary hospitals in Shanghai, China.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

The present invention is based on the unexpected discovery of a method of predicting PCa risk in an individual, based on an assessment of the individual's genotype at a multiplicity (e.g., any of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 in any combination, or all 33) of the 33 SNPs of Table 1. In some embodiments, the method can include an assessment of an individual's genotype at all 33 SNPs of Table 1. In some embodiments, the method can also include an assessment of an individual's genotype at any SNP site in linkage disequilibrium (LD) with any of the 33 SNPs in Table 1. This method, which is called PCS33, provides a powerful predictor of PCa risk. This predictor out-performs any of the currently available parameters of PCa risk as assessed in a unique study population (Table 2). In addition, this predictor can improve the ability of a collection of currently available parameters to predict any PCa risk. Furthermore, this test can be used alone, to identify higher risk individuals who wish to pursue PCa screening or together with established predictors to identify men who may warrant an initial or repeat prostate biopsy. The output of the test can be a cumulative relative risk (CRR, an estimated risk based on the individual's genotype at a multiplicity, in any combination, of any multiplicity of these 33 SNPs, including all 33 SNPs, which is a relative risk based on genotype with respect to the general population), a percentile risk (risk level in percentile in the distribution of the population risk to PCa), absolute risk (risk of PCa over time), or PCa risk score (probability of being diagnosed with PCa as determined by a logistic regression model). There is no true normal value for this test, which allows for the patient or treating physician to determine the risk level which is clinically meaningful to that particular individual. Risk in the general population can be determined, for example, from such sources as surveillance, epidemiology and end results (SEER) information, available on the internet at seer.cancer.gov.

Thus, in one aspect, the present invention provides a method of assessing a subject's risk of having or developing prostate cancer by carrying out an assessment of the subject's genotype at all of the 33 SNP sites or a multiplicity, in any combination, of the 33 SNP sites listed in Table 1 (e.g., a PCS33 risk assessment) according to the methods described herein.

In some embodiments, the PCS33 risk assessment can be used by itself to predict a subject's risk for PCa, which may direct the subject's desire to pursue PCa screening or alter the frequency of PCa screening. This assessment can also be used to identify a subject for whom an initial prostate biopsy or a repeat biopsy is indicated, including for example, a subject who has previously had a negative prostate biopsy and/or a subject for whom no other parameters indicate that an initial prostate biopsy or repeat prostate biopsy should be conducted. Thus, the methods of this invention can further comprise the step of conducting a prostate biopsy (i.e., an initial prostate biopsy or a repeat prostate biopsy) on a subject of this invention.

In further embodiments, the PCS33 risk assessment can be used in combination with known clinical variables (prostate specific antigen (PSA), free to total PSA ratio, age, and/or family history) to predict a subject's risk for PCa. This may help urologists and their patients decide whether to pursue prostate biopsy in men who have never had a prior prostate biopsy and/or who are not considered men for whom an initial prostate biopsy or repeat prostate biopsy would be indicated according to standard practice.

In yet further embodiments, the PCS33 risk assessment can be used in combination with known clinical variables following negative prostate biopsy (prostate volume, number of previous biopsy cores, PSA, free to total PSA ratio, age, and/or family history) to predict a subject's risk for PCa. This may help urologists and their patients decide whether to pursue repeat prostate biopsy in men who have had a prior negative prostate biopsy.

Thus, in one embodiment, the present invention provides a method of identifying a subject for whom a prostate biopsy is indicated, comprising: a) determining, from a nucleic acid sample obtained from the subject, a genotype for the subject at a plurality of biallelic polymorphic loci, wherein each of said plurality has an associated allele and an unassociated allele, wherein the genotype is selected from the group consisting of homozygous for the associated allele, heterozygous, and homozygous for the unassociated allele; b) calculating a genetic risk score (GRS) for the subject based on the genotype determined in step (a); and c) analyzing the GRS of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated. This method can further comprise the step of d) performing a prostate biopsy on the subject identified as a subject for whom a prostate biopsy is indicated according to step (c).

The present invention also provides a method of determining whether to perform a prostate biopsy on a subject, comprising: a) determining, from a nucleic acid sample obtained from the subject, a genotype for the subject at a plurality of biallelic polymorphic loci, wherein each of said plurality has an associated allele and an unassociated allele, wherein the genotype is selected from the group consisting of homozygous for the associated allele, heterozygous, and homozygous for the unassociated allele; b) calculating a genetic risk score (GRS) for the subject based on the genotype determined in step (a); c) analyzing the GRS of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated; d) performing a prostate biopsy on the subject if the subject is identified as a subject for whom a prostate biopsy is indicated according to step (c); and e) not performing a prostate biopsy on the subject if the subject is not identified as a subject for whom a prostate biopsy is indicated according to step (c).

Additionally provided herein is a method of identifying a subject for whom a prostate biopsy is indicated, comprising: a) determining, from a nucleic acid sample obtained from the subject, a genotype for the subject at a plurality of biallelic polymorphic loci, wherein each of said plurality has an associated allele and an unassociated allele, wherein the genotype is selected from the group consisting of homozygous for the associated allele, heterozygous, and homozygous for the unassociated allele; b) calculating a genetic risk score (GRS) for the subject based on the genotype determined in step (a); c) determining, from a sample obtained from the subject, a p2PSA level, a free PSA (fPSA) level and a total PSA (tPSA) level for the subject; d) calculating a prostate health index (PHI) for the subject based on the p2PSA level, fPSA level and tPSA level determined in step (c); and c) analyzing the GRS and PHI of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated. This method can further comprise the step of d) performing a prostate biopsy on the subject identified as a subject for whom a prostate biopsy is indicated according to step (c).

Furthermore, the present invention provides a method of determining whether to perform a prostate biopsy on a subject, comprising: a) determining, from a nucleic acid sample obtained from the subject, a genotype for the subject at a plurality of biallelic polymorphic loci, wherein each of said plurality has an associated allele and an unassociated allele, wherein the genotype is selected from the group consisting of homozygous for the associated allele, heterozygous, and homozygous for the unassociated allele; b) calculating a genetic risk score (GRS) for the subject based on the genotype determined in step (a); c) determining, from a sample obtained from the subject, a p2PSA level, a free PSA (fPSA) level and a total PSA (tPSA) level for the subject; d) calculating a prostate health index (PHI) for the subject based on the p2PSA level, fPSA level and tPSA level determined in step (c); c) analyzing the GRS and PHI of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated; d) performing a prostate biopsy on the subject if the subject is identified as a subject for whom a prostate biopsy is indicated according to step (c); and e) not performing a prostate biopsy on the subject if the subject is not identified as a subject for whom a prostate biopsy is indicated according to step (c).

The present invention further provides a method of identifying a subject for whom a prostate biopsy is indicated, comprising: a) determining, from a sample obtained from the subject, a p2PSA level, a free PSA (fPSA) level and a total PSA (tPSA) level for the subject; b) calculating a prostate health index (PHI) for the subject based on the p2PSA level, fPSA level and tPSA level determined in step (a); c) analyzing the PHI of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated; and d) performing a prostate biopsy on the subject identified as a subject for whom a prostate biopsy is indicated according to step (c).

The present invention additionally provides a method of determining whether to perform a prostate biopsy on a subject, comprising: a) determining, from a sample obtained from the subject, a p2PSA level, a free PSA (fPSA) level and a total PSA (tPSA) level for the subject; b) calculating a prostate health index (PHI) for the subject based on the p2PSA level, fPSA level and tPSA level determined in step (a); c) analyzing the PHI of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated; d) performing a prostate biopsy on the subject if the subject is identified as a subject for whom a prostate biopsy is indicated according to step (c); and e) not performing a prostate biopsy on the subject if the subject is not identified as a subject for whom a prostate biopsy is indicated according to step (c).

In the methods of this invention, the plurality of biallelic polymorphic loci can be a multiplicity, in any combination, of the single nucleotide polymorphisms of Table 1. In some embodiments, the plurality of biallelic polymorphic loci is the 33 single nucleotide polymorphisms of Table 1.

In the methods of this invention, the plurality of biallelic polymorphic loci can be a multiplicity (e.g., 2, 3, 4, 5, 6, 7, 8, 9, 10, 11, 12 or 13), in any combination, of the single nucleotide polymorphisms of Table 10. In some embodiments, the plurality of biallelic polymorphic loci is the 13 single nucleotide polymorphisms of Table 10.

In some embodiments of the methods of this invention, the subject has a family history of prostate cancer and in some embodiments of the methods of this invention, the subject has a prior negative prostate biopsy. In some embodiments, the subject has a prior positive prostate biopsy and in some embodiments, the subject has had no prior prostate biopsy.

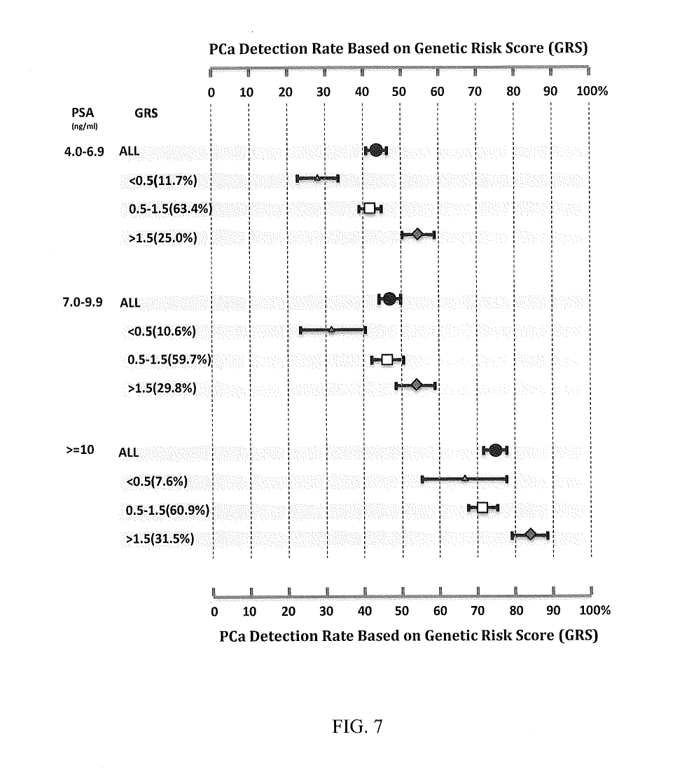

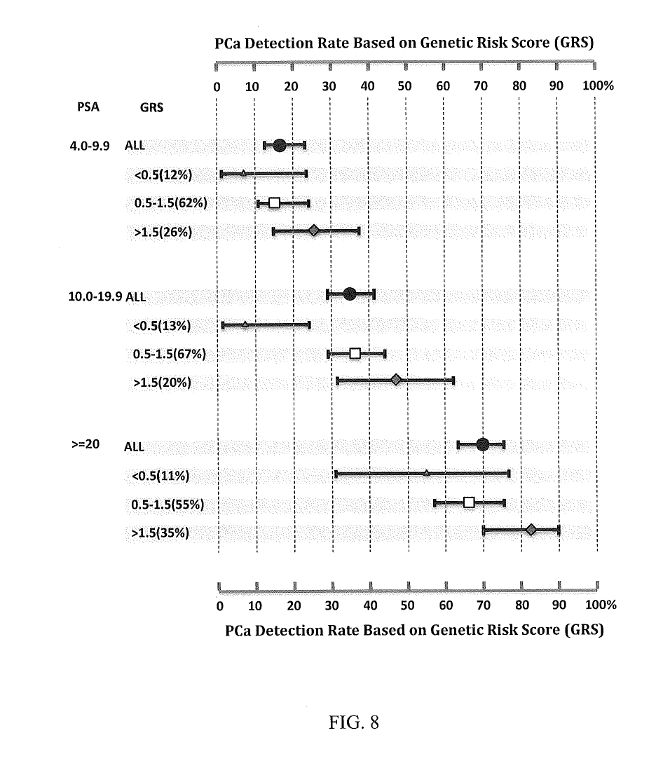

The present invention further provides a chart for visualization of a prostate cancer detection rate for a subject. Thus, in one embodiment, the present invention provides a chart for determining prostate cancer detection rate as a percentile value, said chart comprising: a) a first region comprising a first, second and third prostate specific antigen (PSA) value; b) a second region, adjacent to each respective first, second and third PSA value of the first region, comprising a first, second and third genetic risk score (GRS) value and an average detection rate value (ALL), wherein the first, second and third GRS values are the same for each of the respective PSA values to which the first, second and third GRS values are adjacent; c) a third region, adjacent to the second region, comprising a reference bar to show a prostate cancer detection rate in percentiles ranging from 1% to 100%; and d) a fourth region comprising a grid, aligned below the third region and in parallel with the first, second and third GRS values and the ALL value of the second region for each of the respective first, second and third PSA values of the first region, showing a percentile value that specifies a prostate cancer detection rate for each of the first, second and third GRS values and the ALL value in the second region for each of the first, second and third PSA values in the first region. As two nonlimiting examples, see FIGS. 7 and 8.

In some embodiments, the chart described above can have a first PSA value of 4.0-6.9; a second PSA value of 7.0-9.9; and a third PSA value of ≧10. In some embodiments, the chart can have a first PSA value of 4.0-9.9, a second PSA value of 10.0-19.9; and a third PSA value of ≧20.

In one embodiment, the present invention provides a chart wherein the prostate cancer detection rate is in a range of about 41% to about 47% for a PSA value of 4.0-6.9; the prostate cancer detection rate is in a range of about 22% to about 33% for a PSA value of 4.0-6.9 and a GRS of <0.5; the prostate cancer detection is in a range of about 38% to about 45% for a PSA value of 4.0-6.9 and a GRS of 0.5-1.5; the prostate cancer detection rate is in a range of about 50% to about 59% for a PSA value of 4.0-6.9 and a GRS of >1.5; the prostate cancer detection rate is in a range of about 44% to about 50% for a PSA value of 7.0-9.9; the prostate cancer detection rate is in a range of about 23% to about 41% for a PSA value of 7.0-9.9 and a GRS of <0.5; the prostate cancer detection rate is in a range of about 42% to about 51% for a PSA value of 7.0-9.9 and a GRS of 0.5-1.5; the prostate cancer detection rate is in a range of about 48% to about 59% for a PSA value of 7.0-9.9 and a GRS of >1.5; the prostate cancer detection rate is in a range of about 72% to about 77% for a PSA value of ≧10; the prostate cancer detection rate is in a range of about 55% to about 77% for a PSA value of ≧10 and a GRS of, 0.5; the prostate cancer detection rate is in a range of about 66% to about 75% for a PSA value of ≧10 and a GRS of 0.5-1.5; and a prostate cancer detection rate is in a range of about 78% to about 88% for a PSA value of ≧10 and a GRS of >1.5. This chart is shown in FIG. 7.

In an additional embodiment, the present invention provides a chart wherein the prostate cancer detection rate is in a range of about 13% to about 23% for a PSA value of 4.0-9.9; the prostate cancer detection rate is in a range of about 1% to about 24% for a PSA value of 4.0-9.9 and a GRS of <0.5; the prostate cancer detection is in a range of about 11% to about 24% for a PSA value of 4.0-9.9 and a GRS of 0.5-1.5; the prostate cancer detection rate is in a range of about 14% to about 37% for a PSA value of 4.0-9.9 and a GRS of >1.5; the prostate cancer detection rate is in a range of about 28% to about 42% for a PSA value of 10.0-19.9; the prostate cancer detection rate is in a range of about 1% to about 24% for a PSA value of 10.0-19.9 and a GRS of <0.5; the prostate cancer detection rate is in a range of about 28% to about 45% for a PSA value of 10.0-19.9 and a GRS of 0.5-1.5; the prostate cancer detection rate is in a range of about 31% to about 63% for a PSA value of 10.0-19.9 and a GRS of >1.5; the prostate cancer detection rate is in a range of about 63% to about 75% for a PSA value of ≧20; the prostate cancer detection rate is in a range of about 31% to about 78% for a PSA value of ≧20 and a GRS of <0.5; the prostate cancer detection rate is in a range of about 56% to about 76% for a PSA value of ≧20 and a GRS of 0.5-1.5; and a prostate detection rate is in a range of about 69% to about 90% for a PSA value of ≧20 and a GRS of >1.5. This chart is shown in FIG. 8.

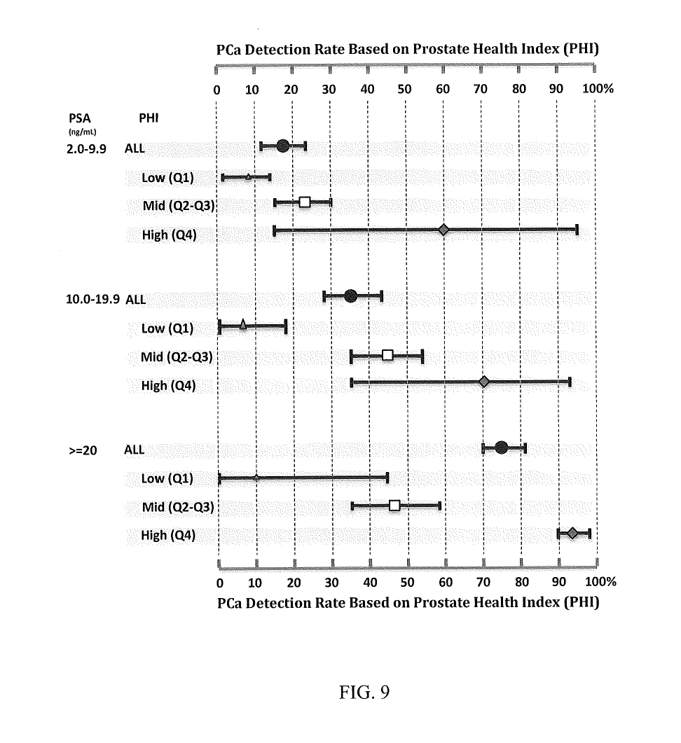

Also provided herein is a chart for determining prostate cancer detection rate as a percentile value, said chart comprising: a) a first region comprising a first, second and third prostate specific antigen (PSA) value; b) a second region, adjacent to each respective first, second and third PSA value of the first region, comprising a first, second and third prostate health index (PHI) value and an average detection rate value (ALL), wherein the first, second and third PHI values are the same for each of the respective PSA values to which the first, second and third PHI values are adjacent; c) a third region, adjacent to the second region, comprising a reference bar to show a prostate cancer detection rate in percentiles ranging from 1% to 100%; and d) a fourth region comprising a grid, aligned below the third region and in parallel with the first, second and third PHI values and the ALL value of the second region for each of the respective first, second and third PSA values of the first region, showing a percentile value that specifies a prostate cancer detection rate for each of the first, second and third PHI values and the ALL value in the second region for each of the first, second and third PSA values in the first region. As a nonlimiting example, see FIG. 9.

The chart described in the paragraph above can be a chart wherein the prostate cancer detection rate is in a range of about 12% to about 23% for a PSA value of 2.0-9.9; the prostate cancer detection rate is in a range of about 2% to about 13% for a PSA value of 2.0-9.9 and a low (Q1) PHI; the prostate cancer detection is in a range of about 15% to about 30% for a PSA value of 2.0-9.9 and a mid (Q2-Q3) PHI; the prostate cancer detection rate is in a range of about 15% to about 95% for a PSA value of 2.0-9.9 and a high (Q4) PHI; the prostate cancer detection rate is in a range of about 27% to about 43% for a PSA value of 10.0-19.9; the prostate cancer detection rate is in a range of about 1% to about 18% for a PSA value of 10.0-19.9 and a low (Q1) PHI; the prostate cancer detection rate is in a range of about 35% to about 54% for a PSA value of 10.0-19.9 and a mid (Q2-Q3) PHI; the prostate cancer detection rate is in a range of about 35% to about 93% for a PSA value of 10.0-19.9 and a high (Q4) PHI; the prostate cancer detection rate is in a range of about 70% to about 82% for a PSA value of ≧20, independent; the prostate cancer detection rate is in a range of about 1% to about 44% for a PSA value of ≧20 and a low (Q1) PHI; the prostate cancer detection rate is in a range of about 35% to about 58% for a PSA value of ≧20 and a mid (Q2-Q3) PHI; and a prostate detection rate is in a range of about 90% to about 97% for a PSA value of ≧20 and a high (Q4) PHI. This chart is shown in FIG. 9.

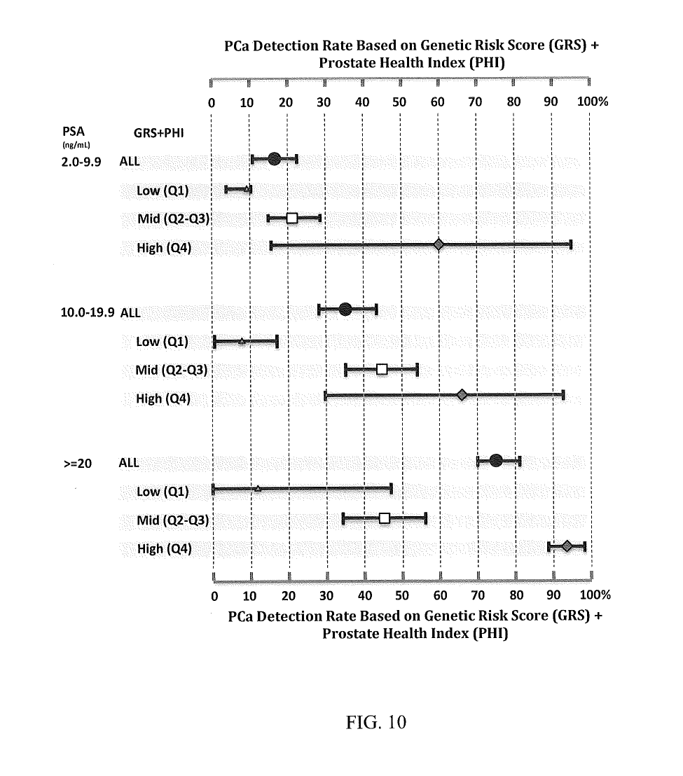

In yet further embodiments, the present invention provides a chart for determining prostate cancer detection rate as a percentile value, said chart comprising: a) a first region comprising a first, second and third prostate specific antigen (PSA) value; b) a second region, adjacent to each respective first, second and third PSA value of the first region, comprising a first, second and third combined prostate health index (PHI) and genetic risk score (GRS) value (GRS+PHI) and an average detection rate value (ALL), wherein the first, second and third GRS+PHI values are the same for each of the respective PSA values to which the first, second and third GRS+PHI values are adjacent; c) a third region, adjacent to the second region, comprising a reference bar to show a prostate cancer detection rate in percentiles ranging from 1% to 100%; and d) a fourth region comprising a grid, aligned below the third region and in parallel with the first, second and third GRS+PHI values and the ALL value of the second region for each of the respective first, second and third PSA values of the first region, showing a percentile value that specifies a prostate cancer detection rate for each of the first, second and third GRS=+PHI values and the ALL value in the second region for each of the first, second and third PSA values in the first region. As a nonlimiting example, see FIG. 10.

The chart described in the above paragraph can be a chart wherein the prostate cancer detection rate is in a range of about 11% to about 23% for a PSA value of 2.0-9.9; the prostate cancer detection rate is in a range of about 4% to about 11% for a PSA value of 2.0-9.9 and a low (Q1) GRS+PHI; the prostate cancer detection is in a range of about 14% to about 28% for a PSA value of 2.0-9.9 and a mid (Q2-Q3) GRS+PHI; the prostate cancer detection rate is in a range of about 15% to about 95% for a PSA value of 2.0-9.9 and a high (Q4) GRS+PHI; the prostate cancer detection rate is in a range of about 27% to about 43% for a PSA value of 10.0-19.9; the prostate cancer detection rate is in a range of about 1% to about 17% for a PSA value of 10.0-19.9 and a low (Q1) GRS+PHI; the prostate cancer detection rate is in a range of about 35% to about 54% for a PSA value of 10.0-19.9 and a mid (Q2-Q3) GRS+PHI; the prostate cancer detection rate is in a range of about 29% to about 93% for a PSA value of 10.0-19.9 and a high (Q4) GRS+PHI; the prostate cancer detection rate is in a range of about 70% to about 82% for a PSA value of ≧20; the prostate cancer detection rate is in a range of about 0% to about 47% for a PSA value of ≧20 and a low (Q1) GRS+PHI; the prostate cancer detection rate is in a range of about 34% to about 56% for a PSA value of ≧20 and a mid (Q2-Q3) GRS+PHI; and a prostate detection rate is in a range of about 88% to about 97% for a PSA value of ≧20 and a high (Q4) GRS+PHI. This chart is shown in FIG. 10.

As used herein, a “reference value” can be a threshold value for determining whether to perform a prostate biopsy on a subject and such a reference value as applied to the methods of this invention can be about 30%, 35%, 40%, 45%, 50%, 55%, 60%, 70% or 75%, including any values within this range not explicitly recited herein. A reference value as used herein can also be a value that is about 1%, 2%, 3%, 4%, 5%, 6%, 7%, 8%, 9%, 10%, 15%, 20%, 25%, 30%, 40%, 50%, 60%, 70%, 80%, 90% or 10)% above the ALL value shown in the chart.

The step of determining includes manipulating a fluid or tissue sample obtained from the subject to extract nucleic acid of the subject from the sample in a form that allows for the nucleotide sequence of the nucleic acid to be identified. The method can further comprise the step of carrying out the prostate biopsy on a subject for whom a prostate biopsy is indicated according to the steps of the method as described herein.

The genetic risk score (GRS) calculation is described in some embodiments as follows: a weighted genetic score is calculated for each subject based on the genotypes at 33 prostate cancer risk-associated SNPs and weighted by the respective odds ratio (OR) of each of these SNPs derived from an external study using a method described by Pharoah et al (“Polygenes, risk prediction, and targeted prevention of breast cancer” N Engl J Med 358:2796-2803 (2008)) Briefly, 1) the allelic OR for each SNP was obtained from an external study, 2) the genotypic OR of each SNP was estimated from the allelic OR assuming a multiplicative model, 3) the risk relative to the average risk in the population was calculated for each genotype based on genotypic OR and genotype frequency in the study population, and 4) genetic score was obtained by multiplying the risks relative to the population of all SNPs. Therefore, a genetic score of 1.0 indicates an average risk in the general population.

The prostate health index (PHI) is calculated based on PHI=(p2PSA/fPSA)×√(tPSA).

The prostate cancer (PCa) detection rate is based on subjects that were positive for PCa based on the particular target group as identified in the chart (e.g., PSA 2.0-10.0 ng/ml and 1st quartile with GRS within that group). This is known as the point estimate and a 95% CI is calculated around that (the black lines). To generate the charts of this invention, the positive detection rate (how many positives within the selected group) is applied relative to the PSA, GRS and PHI values. Thus, the detection rate is the number of PCa positives/total sample population in each target group.

In the methods of this invention, the plurality of biallelic polymorphic loci employed in the methods of this invention is a multiplicity (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33), in any combination, of the 33 single nucleotide polymorphisms of Table 1. In some embodiments, the plurality of biallelic polymorphic loci employed in the methods of this invention is the 33 single nucleotide polymorphisms of Table 1.

The risk assessment provided to the patient subjects and their treating urologist may include any or all of the following.

1. Cumulative relative risk (CRR) to PCa. The CRR to PCa provided to the subject is derived by obtaining the subject's genotype at the 33 SNPs of Table 1 and may in addition include information on clinical parameters should they be available. For the genetic component of the CRR (CRR), allelic odds ratios (ORs) are obtained from meta-analyses which are then used to determine a relative risk to the general population for a particular genotype at a particular SNP for an individual. The CRR based on 33 SNPs or a multiplicity, in any combination, of the 33 SNPs is then generated by multiplying the relative risks for each of the SNPs for a given individual. This is the genetic component of the CRR to PCa presented to the subject and represents the fold increase in PCa risk compared to the general population. A similar analysis may be performed including the ORs and relative risks for each available clinical parameter based on the outlined study population and then can be used with the genetic component to provide an overall CRR to PCa.

2. Percentile risk to PCa. The percentile risk is generated by determining the risk level in percentile in the distribution of population relative risk for PCa.

3. Absolute risk to PCa. Absolute risk is determined by taking into consideration the CRR and incidence and mortality rates from PCa and mortality due to other causes. This describes the PCa risk over time and for the purposes of this invention, represents the lifetime risk of PCa.

4. PCa risk score. PCa risk score is another means to measure the probability of being diagnosed with PCa. It does not take into consideration time or population parameters such as disease incidence or mortality rates. It is generated by fitting the CRR from the genetic component alone or in combination with other predictors (including genetic score, PSA, F/T PSA ratio, family history of PCa, age), into a logistic regression model.

### DEFINITIONS

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

As used herein, the term “prostate cancer” or “PCa” describes an uncontrolled (malignant) growth of cells originating from the prostate gland, which is located at the base of the urinary bladder and is responsible for helping control urination as well as forming part of the semen. Symptoms of prostate cancer can include, but are not limited to, urinary problems (e.g., not being able to urinate; having a hard time starting or stopping the urine flow; needing to urinate often, especially at night; weak flow of urine; urine flow that starts and stops; pain or burning during urination), difficulty having an erection, blood in the urine and/or semen, and/or frequent pain in the lower back, hips, and/or upper thighs.

As used herein, the term “aggressive prostate cancer” means prostate cancer that is poorly differentiated, having a Gleason grade of 7 or above and an “indolent prostate cancer” having a Gleason grade of 6. The Gleason grading system is the most commonly used method for grading PCa.

All the SNP positions described herein are based on Build 36.

Also as used herein, “linked” describes a region of a chromosome that is shared more frequently in family members or members of a population manifesting a particular phenotype and/or affected by a particular disease or disorder, than would be expected or observed by chance, thereby indicating that the gene or genes or other identified marker(s) within the linked chromosome region contain or are associated with an allele that is correlated with the phenotype and/or presence of a disease or disorder (e.g., aggressive PCa), or with an increased or decreased likelihood of the phenotype and/or of the disease or disorder. Once linkage is established, association studies can be used to narrow the region of interest or to identify the marker (e.g., allele or haplotype) correlated with the phenotype and/or disease or disorder.

Furthermore, as used herein, the term “linkage disequilibrium” or “LD” refers to the occurrence in a population of two or more (e.g., 3, 4, 5, 6, 7, 8, 9, 10, etc.) linked alleles at a frequency higher or lower than expected on the basis of the gene frequencies of the individual genes. Thus, linkage disequilibrium describes a situation where alleles occur together more often than can be accounted for by chance, which indicates that the two or more alleles are physically close on a DNA strand.

The term “genetic marker” or “polymorphism” as used herein refers to a characteristic of a nucleotide sequence (e.g., in a chromosome) that is identifiable due to its variability among different subjects (i.e., the genetic marker or polymorphism can be a single nucleotide polymorphism, a restriction fragment length polymorphism, a microsatellite, a deletion of nucleotides, an addition of nucleotides, a substitution of nucleotides, a repeat or duplication of nucleotides, a translocation of nucleotides, and/or an aberrant or alternate splice site resulting in production of a truncated or extended form of a protein, etc., as would be well known to one of ordinary skill in the art).

A “single nucleotide polymorphism” (SNP) in a nucleotide sequence is a genetic marker that is polymorphic for two (or in some case three or four) alleles. SNPs can be present within a coding sequence of a gene, within noncoding regions of a gene and/or in an intergenic (e.g., intron) region of a gene. A SNP in a coding region in which both forms lead to the same polypeptide sequence is termed synonymous (i.e., a silent mutation) and if a different polypeptide sequence is produced, the alleles of that SNP are non-synonymous. SNPs that are not in protein coding regions can still have effects on gene splicing, transcription factor binding and/or the sequence of non-coding RNA.

The SNP nomenclature provided herein refers to the official Reference SNP (rs) identification number as assigned to each unique SNP by the National Center for Biotechnological Information (NCBI), which is available in the GenBank® database.

In some embodiments, the term genetic marker is also intended to describe a phenotypic effect of an allele or haplotype, including for example, an increased or decreased amount of a messenger RNA, an increased or decreased amount of protein, an increase or decrease in the copy number of a gene, production of a defective protein, tissue or organ, etc., as would be well known to one of ordinary skill in the art.

An “allele” as used herein refers to one of two or more alternative forms of a nucleotide sequence at a given position (locus) on a chromosome. An allele can be a nucleotide present in a nucleotide sequence that makes up the coding sequence of a gene and/or an allele can be a nucleotide in a non-coding region of a gene (e.g., in a genomic sequence). A subject's genotype for a given gene is the set of alleles the subject happens to possess. As noted herein, an individual can be heterozygous or homozygous for any allele of this invention.

Also as used herein, a “haplotype” is a set of alleles on a single chromatid that are statistically associated. It is thought that these associations, and the identification of a few alleles of a haplotype block, can unambiguously identify all other alleles in its region. The term “haplotype” is also commonly used to describe the genetic constitution of individuals with respect to one member of a pair of allelic genes; sets of single alleles or closely linked genes that tend to be inherited together.

The terms “increased risk” and “decreased risk” as used herein define the level of risk that a subject has of developing prostate cancer, as compared to a control subject that does not have the polymorphisms and alleles of this invention in the control subject's nucleic acid.

A sample of this invention can be any sample containing nucleic acid of a subject, as would be well known to one of ordinary skill in the art. Nonlimiting examples of a sample of this invention include a cell, a body fluid, a tissue, biopsy material, a washing, a swabbing, etc., as would be well known in the art.

A subject of this invention is any animal that is susceptible to prostate cancer as defined herein and can include, for example, humans, as well as animal models of prostate cancer (e.g., rats, mice, dogs, nonhuman primates, etc.). In some aspects of this invention, the subject can be Caucasian (e.g., white; European-American; Hispanic), as well as of black African ancestry (e.g., black; African American; African-European; African-Caribbean, etc.) or Asian. In further aspects of this invention, the subject can have a family history of prostate cancer or aggressive prostate cancer (e.g., having at least one first degree relative having or diagnosed with prostate cancer or aggressive prostate cancer) and in some embodiments, the subject does not have a family history of prostate cancer or aggressive prostate cancer. Additionally a subject of this invention can have a diagnosis of prostate cancer in certain embodiments and in other embodiments, a subject of this invention does not have a diagnosis of prostate cancer. In yet further embodiments, the subject of this invention can have an elevated prostate-specific antigen (PSA) level and in other embodiments, the subject of this invention can have a normal or non-elevated PSA level. In some embodiments, the PSA level of the subject may not be known and/or has not been measured.

As used herein, “nucleic acid” encompasses both RNA and DNA, including cDNA, genomic DNA, mRNA, synthetic (e.g., chemically synthesized) DNA and chimeras, fusions and/or hybrids of RNA and DNA. The nucleic acid can be double-stranded or single-stranded. Where single-stranded, the nucleic acid can be a sense strand or an antisense strand. In some embodiments, the nucleic acid can be synthesized using oligonucleotide analogs or derivatives (e.g., inosine or phosphorothioate nucleotides, etc.). Such oligonucleotides can be used, for example, to prepare nucleic acids that have altered base-pairing abilities or increased resistance to nucleases.

An “isolated nucleic acid” is a nucleotide sequence that is not immediately contiguous with nucleotide sequences with which it is immediately contiguous (one on the 5′ end and one on the 3′ end) in the naturally occurring genome of the organism from which it is derived or in which it is detected or identified. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” can refer to a nucleic acid or polypeptide that is substantially free of cellular material, viral material, and/or culture medium (e.g., when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

The term “oligonucleotide” refers to a nucleic acid sequence of at least about five nucleotides to about 500 nucleotides (e.g. 5, 6, 7, 8, 9, 10, 12, 15, 18, 20, 21, 22, 25, 30, 35, 40, 45, 50, 55, 60, 65, 70, 75, 80, 85, 90, 100, 125, 150, 175, 200, 250, 300, 350, 400, 450 or 500 nucleotides). In some embodiments, for example, an oligonucleotide can be from about 15 nucleotides to about 30 nucleotides, or about 20 nucleotides to about 25 nucleotides, which can be used, for example, as a primer in a polymerase chain reaction (PCR) amplification assay and/or as a probe in a hybridization assay or in a microarray. Oligonucleotides of this invention can be natural or synthetic, e.g., DNA, RNA, PNA, LNA, modified backbones, etc., as are well known in the art.

The present invention further provides fragments of the nucleic acids of this invention, which can be used, for example, as primers and/or probes. Such fragments or oligonucleotides can be detectably labeled or modified, for example, to include and/or incorporate a restriction enzyme cleavage site when employed as a primer in an amplification (e.g., PCR) assay.

The detection of a polymorphism, genetic marker or allele of this invention can be carried out according to various protocols standard in the art and as described herein for analyzing nucleic acid samples and nucleotide sequences, as well as identifying specific nucleotides in a nucleotide sequence.

For example, nucleic acid can be obtained from any suitable sample from the subject that will contain nucleic acid and the nucleic acid can then be prepared and analyzed according to well-established protocols for the presence of genetic markers according to the methods of this invention. In some embodiments, analysis of the nucleic acid can be carried by amplification of the region of interest according to amplification protocols well known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA), etc.). The amplification product can then be visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, and/or by electrophoresis. Thus, the present invention further provides oligonucleotides for use as primers and/or probes for detecting and/or identifying genetic markers according to the methods of this invention.

In some embodiments of this invention, detection of an allele or combination of alleles of this invention can be carried out by an amplification reaction and single base extension. In particular embodiments, the product of the amplification reaction and single base extension is spotted on a silicone chip.

In yet additional embodiments, detection of an allele or combination of alleles of this invention can be carried out by matrix-assisted laser desorption/ionization-time of flight mass spectrometry (MALDI-TOF-MS).

It is further contemplated that the detection of an allele or combination of alleles of this invention can be carried out by various methods that are well known in the art, including, but not limited to nucleic acid sequencing, hybridization assay, restriction endonuclease digestion analysis, electrophoresis, and any combination thereof.

The present invention further comprises a kit or kits to carry out the methods of this invention. A kit of this invention can comprise reagents, buffers, and apparatus for mixing, measuring, sorting, labeling, etc, as well as instructions and the like as would be appropriate for genotyping the 33 SNPs of Table 1 in a nucleic acid sample. The kit may further comprise control reagents, e.g., to identify markers for a specific ethnicity or gender.

The present invention is more particularly described in the following examples that are intended as illustrative only since numerous modifications and variations therein will be apparent to those skilled in the art.

### EXAMPLES

### Example 1

### Methods of Genetic Test to Determine Genetic Score

In a hierarchical order, three models were used to predict PCa risk. First, a “genetic marker only” model was used in which 33 SNPs identified by genome wide association studies (GWAS) as associated with PCa risk were included. Second, a “genetic marker+pre-biopsy variable model”; in addition to the 33 SNPs, was used. This model included age, family history, and ratio of baseline free PSA to baseline total PSA. Third, a “genetic+pre-biopsy variable+post-biopsy variable model” was used. In addition to the second model, this third model included baseline prostate volume and number of previous biopsy cores. Each model was used to perform risk assessment, which included estimating various measures of PCa risk, including the cumulative relative risk (CRR), percentile risk, absolute risk, and risk score (i.e., the predicted probability of being diagnosed with PCa as determined by a regression model). The predictive performance of each model is measured by detection rate of PCa during the four years of the REDUCE trial, providing an overall assessment of clinical validity. Detailed methods for estimating these measures of risk are described below.

Odds Ratio (OR) Calculations.

ORs for the 33 SNPs were calculated using external data presented in the literature. ORs for the clinical variables were estimated from the study sample. For the allelic ORs, the best estimates and their confidence intervals (CIs) for the 33 SNPs were obtained using meta-analysis. The details of the meta-analysis are described below. First, if the literature search yielded raw data such as allele counts of case and control, then this information was used for calculating the OR and standard error for each study population. Otherwise, these estimates were calculated using the reported OR and 95% CI. The results from both approaches are statistically comparable. Second, while integrating different study results, the heterogeneity of estimated ORs was assessed across study populations. The Q-statistic (for test of heterogeneity) and 12 statistic (which measures the proportion of total variance in estimated ORs due to heterogeneity) were used. If there was evidence of a high degree of heterogeneity, such as a value of the 12 statistic greater than 50%, then the random effects method was used to calculate the pooled OR and CI. Otherwise, the fixed effects method was used. The fixed effects method weighs each study with the inverse of variance of logarithm of OR, while the random effects method additionally incorporates variance in that weight. Furthermore, the ORs for the demographic and clinical variables were calculated by applying the multiple logistic regression in the present study sample since they were not available from the meta-analysis. Each of the demographic or clinical variables has been categorized with meaningful cut-off points.

Relative Risk (RR) Calculation.

For each of the three genotypes at each SNP, the allelic OR was converted to the RR relative to the general population using the following approach. The average population risk compared to non-carriers was a weighted average of the relative risks of the genotypes. Specifically, the ratio between the average population risk and the risk of non-carriers was estimated by A=P(rr)×OR²+P(wr)×OR+P(ww), where w is the wild type allele, r is the risk allele, and P(ww), P(wr), and P(rr) are the proportions of the population carrying ww, wr, and rr, respectively. RRs for ww, wr, and rr were estimated by 1/A, OR/A, and OR²/A, respectively. The corresponding confidence intervals were estimated accounting for variability of estimates of OR. Furthermore, the RRs for the clinical variables were calculated in a similar manner. The ratio between the average population risk and the risk of the reference group was estimated by summing over the product of frequency of each category and the corresponding OR. Then the RR was calculated accordingly.

Measures of Risk.

Cumulative relative risk (CRR), percentile risk to PCa, absolute risk, and risk score were used as measures of risk to PCa in this study. To estimate cumulative relative risk, the controls were assumed to be a random sample from the general population. For the genetic only model, a multiplicative model was used, in which RRs for each of the SNPs for a given individual were multiplied. For the other two models, the CRR relative to the population was derived by combining the RRs for the 33 SNPs as well as RRs for the clinical variables of the individual by simple multiplication. The percentile risk to PCa was generated by determining the risk level in terms of percentile within the distribution of population CRR.

The absolute risk for each individual was then estimated based on the overall CRR, relative to the population (r(a, x)), the incidence rate of PCa in the general population (λ₀(x)), and the all-cause mortality rate excluding PCa in the United States (μ₀(x)). Specifically, assuming the mortality data are known without error and do not vary with the risk factors in this model, mortality data from the National Center of Health Statistics was used to estimate the mortality rate from non-PCa causes. Let F (a, t) denote the probability that one survives until age t without developing PCa. Then F(a,t)=exp {−∫ₐ^{t}[r(a,x)λ₀(x)+μ₀(x)]dx}. The probability that one develops PCa in a small interval equals the probability of his/her disease free survival until age t times the conditional probability of developing PCa by age t+Δt given that one was disease free at age t. This probability, absolute risk, is conditioned on the fact that one has not developed PCa by age a. The corresponding CIs can be calculated accounting for the variability of estimates of relative risks and of risk factor distributions.

The risk score was the predicted value of PCa risk from a logistic regression model with the CRR from the genetic component alone or in combination with other clinical variables as the covariate. It is calculated as

$$\frac{exp ( {\hat{β}}_{0} + {\hat{β}}_{1} X )}{1 + exp ( {\hat{β}}_{0} + {\hat{β}}_{1} X )} ,$$

where X is the relative risk, \hat{β}₀ and \hat{β}₁ are regression coefficient estimates for the intercept and relative risk, respectively. The corresponding CI can be calculated by converting the CIs for the linear combination of the estimated coefficients and the values of the relative risk (i.e., \hat{β}₀+\hat{β}₁X).

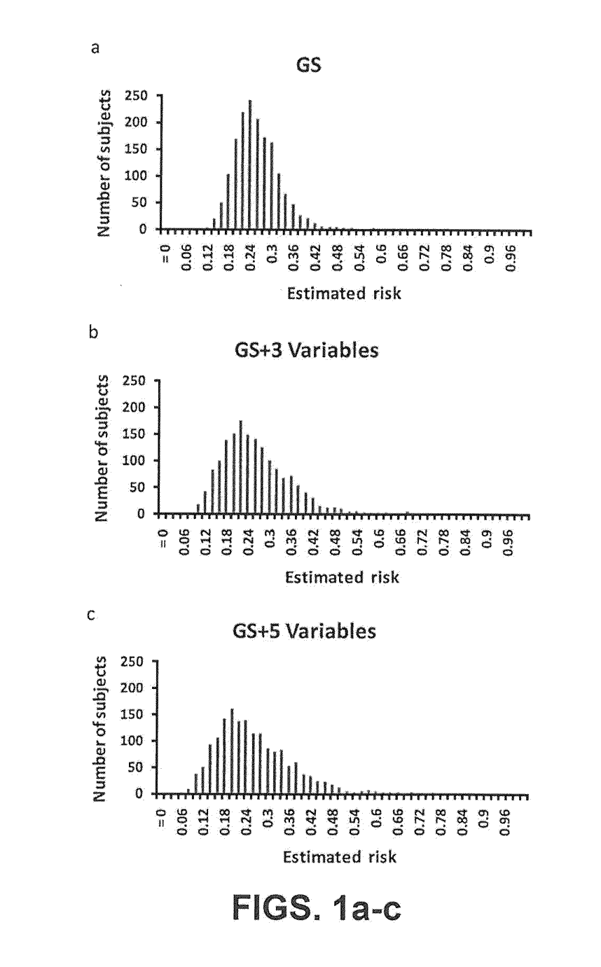

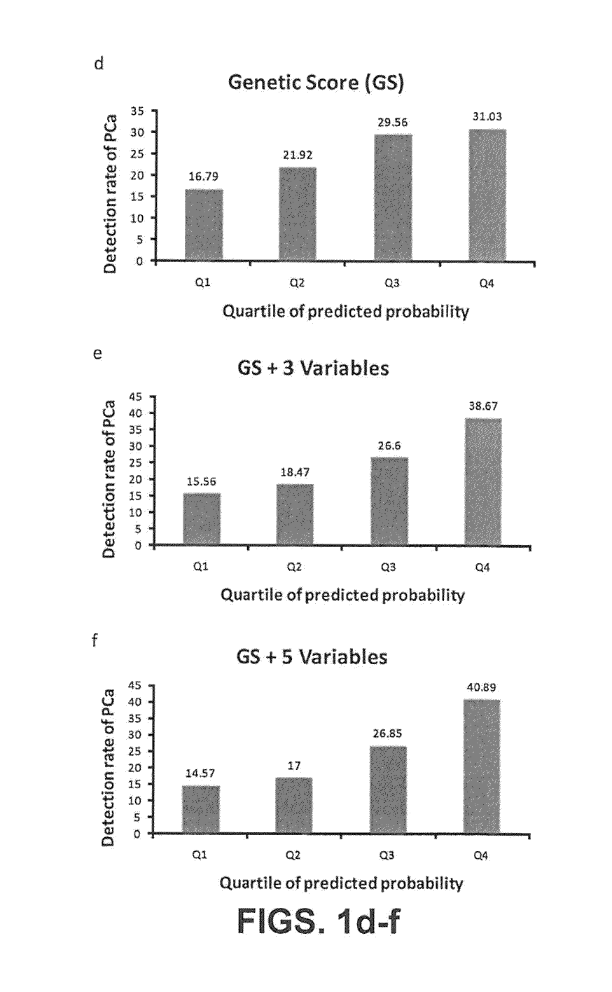

The distributions of risk score among the REDUCE study subjects are presented in FIGS. 1a-c for genetic marker only, genetic marker+pre-biopsy variable model,” and “genetic+pre-biopsy variable+post-biopsy variable model,” respectively.

Detection Rate.

In order to assess clinical validity, the detection rate of PCa during the 4-year study of the REDUCE study was calculated for each model to measure their predictive performance. The sample was divided equally into quartiles based on the estimated risk of risk. Detection rate was then calculated as the proportion of positive biopsies in each quartile. To obtain unbiased estimates, four-fold cross-validation was used to calculate detection rates. Four-fold cross validation randomly divides the data into four (roughly) equal subsets and repeatedly uses three subsets for model fitting (training) and the remaining subset for validation (testing), in order to calculate the detection rate. This process was repeated until each of the four subsets had been used exactly once as validation data, after which detection rates were averaged across results from each of the four validation sets. All of the detection rates in the testing samples of four-fold cross validation were reported except for the genetic model, because the genetic score was calculated based on external OR estimates of the 33 SNPs. The observed detection rates of PCa during the four-year REDUCE study are presented in FIGS. 1d-f for men at each quartile of estimated risk based on genetic marker only, genetic marker+pre-biopsy variable model,” and “genetic+pre-biopsy variable+post-biopsy variable model,” respectively.

In some embodiments of this invention, a genetic score that places an individual in the 50th percentile or greater is indicative of increased risk of PCa. An absolute risk value of greater than about 0.13 is indicative of increased risk of PCa. A CRR of greater than 1.0 is indicative of increased risk of PCa. A genetic score that places an individual below the 50th percentile is indicative of decreased risk of PCa. An absolute risk value of less than about 0.13 is indicative of decreased risk of PCa. A CRR of less than 1.0 is indicative of decreased risk of PCa. Increased risk and decreased risk as used herein mean increased or decreased relative to the general population (see, e.g., SEER information at seer.cancer.gov).

Furthermore, a population median risk score can be used as the cutoff for indicating increased or decreased risk (i.e., a risk score above the cutoff indicates increased risk and a risk score below the cutoff indicates decreased risk). This differs for each of the three models. For genetic only model, the cutoff is 0.24, for genetic+pre-biopsy model, the cutoff is 0.23 and for genetic+pre-biopsy+post-biopsy, the cutoff is 0.23.

Increased risk and decreased risk as used herein mean increased or decreased relative to the general population.

### Example 2

### Clinical Utility of Inherited Genetic Markers for the Prediction of Prostate Cancer at Repeat Biopsy: Results from Placebo Arm of the Reduce® Clinical Trial

Background.

The predictive performance of available clinical parameters for prostate cancer (PCa) is limited, particularly following negative prostate biopsy. This study was done to assess the clinical utility of identified PCa risk-associated single nucleotide polymorphisms (SNPs) for PCa prediction in a clinical trial.

Methods.

Subjects included 1,654 men who consented for genetic studies in the placebo arm of the randomized REduction by DUtasteride of Prostate Cancer Events (REDUCE) trial, where all subjects had a negative prostate biopsy at baseline and underwent scheduled prostate biopsies at years 2 and 4. Predictive performance of clinical parameters at baseline, and/or a genetic score based on 33 PCa risk-associated SNPs was evaluated using the area under the receiver operating characteristic curve (AUC) and PCa detection rate.

Findings.

Of the 1,654 men, 410 (25%) were diagnosed with PCa during the four year follow-up. The genetic score based on the 33 SNPs was a highly significant predictor for positive biopsy even after adjusting for known clinical variables (P=3.58×10⁻⁸). Measured by AUC, the genetic score outperformed any individual clinical parameter including prostate-specific antigen (PSA) for PCa risk prediction, and improved the performance of the best combined clinical model consisting of age, family history, free/total PSA ratio, prostate volume, and number of initial biopsy cores. The added value of the genetic score is highlighted by its ability to further differentiate PCa detection rates defined by the best clinical model. The observed PCa detection rate over 4-years was 19.16% higher for men with higher estimated clinical risk/higher genetic score (34.82%) than with lower estimated clinical risk/lower genetic score (15.66%), P=3.3×10⁻¹⁰.

Interpretations.

This clinical trial provides the next level of evidence, that germline markers may be used to supplement existing clinical parameters to better predict outcome of prostate biopsy.

Introduction.

Prostate cancer (PCa) is the most common solid organ malignancy affecting American men and the second leading cause of cancer related death. Approximately one million prostate biopsies are performed yearly in the U.S. The vast majority of these biopsies are performed due to elevated levels of the PCa marker prostate-specific antigen (PSA). However, only a quarter of these biopsies result in a diagnosis of PCa, highlighting the inadequate performance of PSA to predict PCa. Persistently elevated PSA levels and/or other clinical parameters that prompted initial biopsies contribute to stress and anxiety among both patients and their urologists. More relevant approaches employing biomarkers are urgently needed to better determine the need for initial and repeat prostate biopsy.

Recently, more than 30 PCa risk-associated single nucleotide polymorphisms (SNPs) have been discovered from genome-wide association studies (GWAS). These risk-associated SNPs have been consistently replicated in multiple case-control study populations of

European descent. Although each of these SNPs is only moderately associated with PCa risk, a genetic score based on a combination of risk-associated SNPs can be used to identify men at high risk for PCa. These risk-associated SNPs may have broad practical applications because they are common in the general population.

Study Population.

Subjects included 1,654 of the 3,129 (53%) men of European descent in the placebo arm of the randomized, multi-institutional, international, Reduction by DUtasteride of Prostate Cancer Events (REDUCE) study who consented for genetic studies. The characteristics of patients who consented or declined genetic studies are presented in Table 3. The REDUCE study is a randomized double blind chemoprevention trial, examining PCa risk reduction by dutasteride, a dual 5-alpha reductase inhibitor, in a population of men with prior negative prostate biopsy. Eligible men were 50 to 75 years of age, with a serum PSA≧2.5 ng/mL and ≦10 ng/mL (men aged 50-60 years) or ≧3.0 ng/mL and ≦10 ng/mL (men>60 years of age), and had a single, negative prostate biopsy (6-12 cores) within 6 months prior to enrollment (independent of the study). Exclusion criteria included more than one prior prostate biopsy, high-grade prostatic intra-epithelial neoplasia (HG-PIN) or atypical small acinar proliferation (ASAP) on the pre-study entry prostate biopsy assessed by a central pathology laboratory, or a prostate volume greater than 80 cc.

PCa Risk-Associated SNPs, Ancestry Informative Markers (AIMs), and Genotyping.

A panel of 33 PCa risk-associated SNPs was selected from all PCa GWAS reported before December 2009. Each of these SNPs exceeded genome-wide significance levels in their initial reports (P<10⁻⁷) and these associations have been replicated in independent study populations. In addition, 91 SNPs from a panel of 93 AIMs were genotyped to distinguish population groups from major continents. These SNPs were genotyped using the Sequenom MassARRAY platform. One duplicated CEPH (Centre d'Etude du Polymorphisme Humain) sample and two water samples (negative controls) that were blinded to technicians were included in each 96-well plate. The concordance rate between the two genotype calls of the duplicated CEPH sample for all SNPs was 100%.

Statistical Analyses.

Allelic odds ratios (ORs) and 95% confidence intervals (Cis) for each of the 33 SNPs were estimated using an unconditional logistic regression model, adjusting for ethnic structure using the first two principal components, as is standard in genetic association studies. A genetic score (GRS), based on all 33 SNPs and OR estimates from an external meta-analysis, was calculated for each individual. Briefly, a multiplicative model was used to derive genotype relative risks from the external allelic OR. For each of the three genotypes at each SNP, the genotype relative risk was converted to the risk, relative to the population. The overall risk, relative to the population (i.e., genetic score or GRS), was derived by combining the risks, relative to the population, of all SNPs of each individual by simple multiplication.

Chi-square and t-tests were used to compare the differences between groups of subjects for binary variables (family history, digital rectal exam [DRE], and continuous variables (age, PSA measurements, prostate volume, number of cores at pre-study entry biopsy, and genetic score), respectively. Total PSA and genetic score were log transformed to approach a normal distribution.

The AUC of clinical predictors and genetic score, individually and in combination, for predicting PCa was estimated using a logistic regression model. Four-fold cross validation was used to reduce the bias in estimates of AUC. Subjects were randomly divided into four groups. A model was fit to each three-quarter subset of the subjects and tested on the remaining one-quarter subset of subjects, yielding four testing AUCs. Results from 10 runs of four-fold cross validation are reported.

The detection rate of PCa for men at various estimated risk categories was also calculated based on prediction models. Unbiased detection rates were directly estimated for the genetic model, because the genetic score of each individual was calculated based on external OR estimates of the 33 SNPs. For the clinical model, four-fold cross validation was used to obtain unbiased estimates, as described below. Coefficients of variables in the prediction models were estimated from each three-quarter subset of the subjects and used to calculate risk in the remaining one-quarter subset of subjects. Each of these one-quarter subsets of subjects was ranked based on estimated risk and then equally divided into two groups. The PCa detection rate was calculated as the proportion of positive biopsy in each group. Results from 10 runs of four-fold cross validation are reported.

Results.

Among the 1,654 men of European descent who had an initial negative biopsy for PCa and who consented to genetic studies in the placebo arm of the REDUCE trial, 410 men (25%) had a positive prostate biopsy for PCa from scheduled and for-cause biopsies over the four-year study. In a univariate analysis (Table 4), men with positive biopsies differed significantly (P<0.05) from men with negative prostate biopsies for all of the baseline clinical and demographic variables, with the exception of DRE. Significant differences were also observed for genetic risk factors; positive family history of PCa was found in 17% of the men with positive biopsy, compared with 12% of the men with negative biopsy (OR=1.5 [95% CI: 1.09-2.04], P=0.01), and the difference in the genetic score between these two groups was highly significant (P=4.95×10⁻⁹). After adjusting for known PCa risk-associated clinical variables such as age, free/total PSA ratio, number of cores at initial biopsy, and prostate volume using multivariate logistic regression analysis, family history and genetic score remained significantly associated with positive prostate biopsy (P=0.002 and 3.58×10⁻⁸, respectively).

The AUC of these baseline clinical variables and genetic risk factors was calculated, individually and in combination, for predicting positive prostate biopsy during the four-year follow-up. To obtain unbiased estimates of AUC, a four-fold cross validation method was used and results from testing samples are reported. Among individual predictors, the AUC of the genetic score was highest (0.59), followed by prostate volume (0.56), age (0.56), number of cores sampled at pre-study entry biopsy (0.55), free/total PSA ratio (0.54), total PSA (0.54), family history (0.52), and DRE (0.51). When multiple predictors were included in the model simultaneously, the best clinical model included five baseline variables (age, family history, free/total PSA ratio, number of cores at pre-study entry biopsy, and prostate volume), with an AUC of 0.60. When the genetic score was added to this best clinical model, the AUC increased to 0.64.

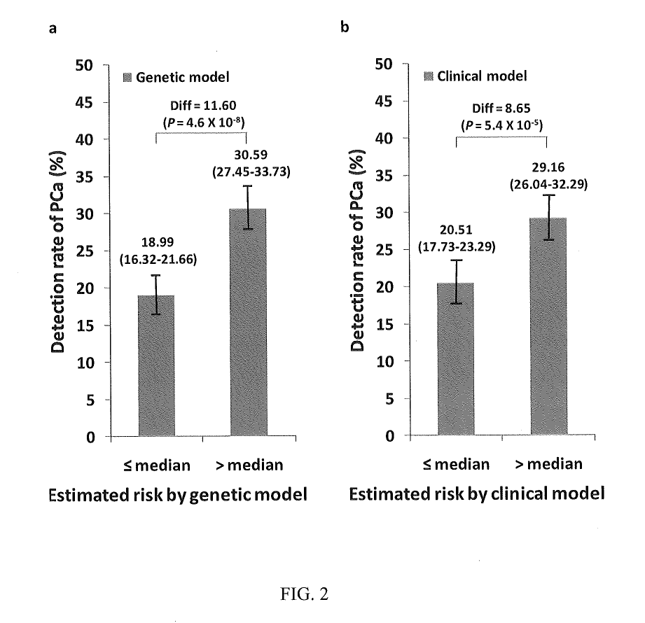

To facilitate the use and interpretation of these models in predicting positive prostate biopsy, the PCa detection rate was calculated during four years for the genetic score model and the best clinical model. Each individual's risk for PCa was estimated using either the genetic score model or the best clinical model, and was classified as being lower or higher risk for PCa (compared to the median risk) under each model. The observed detection rates of PCa for men at different estimated risks under each model are presented in FIGS. 2a-b. Both the genetic model and the best clinical model were able to differentiate detection rate between these two groups of men, although the genetic model performed better. In the genetic model, the observed detection rate was 11.60% higher for men who had higher estimated risk (30.59%) than those with lower estimated risk (18.99%). The difference was highly significant, P=4.6×10⁻⁸. In the best clinical model, the observed detection rate was 8.65% higher for men who had higher estimated risk (29.16%) than those with lower estimated risk (20.51%). The difference was also significant, P=5.4×10⁻⁵.

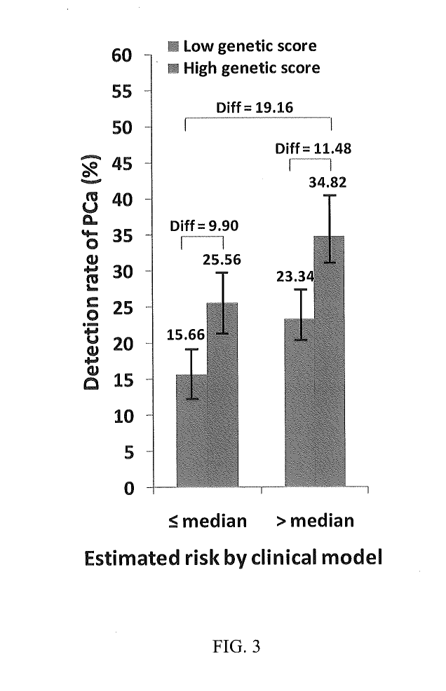

To further examine the value of adding the genetic score to existing clinical parameters in predicting positive prostate biopsy, PCa detection rates were estimated among men who were classified as the same risk based on the best clinical model but having different genetic scores (FIG. 3). The genetic score was able to further differentiate detection rate. For men at lower clinical risk, the detection rate for PCa was 9.90% higher for men whose genetic score was above the median (25.56%) than those below the median (15.66%), P=4.9×10⁻⁴. Similarly, for men at higher clinical risk, the detection rate for PCa was 11.48% higher for men who had higher genetic score (34.82%) than lower genetic score (23.34%), P=3.2×10⁻⁴. Combining the genetic model and the best clinical model, they were able to considerably differentiate detection rate between the extreme groups of men. The detection rate was 19.16% higher for men who have higher estimated clinical risk/higher genetic score (34.82%) than men who had lower estimated clinical risk/lower genetic score (15.66%), P=3.3×10⁻¹⁰.

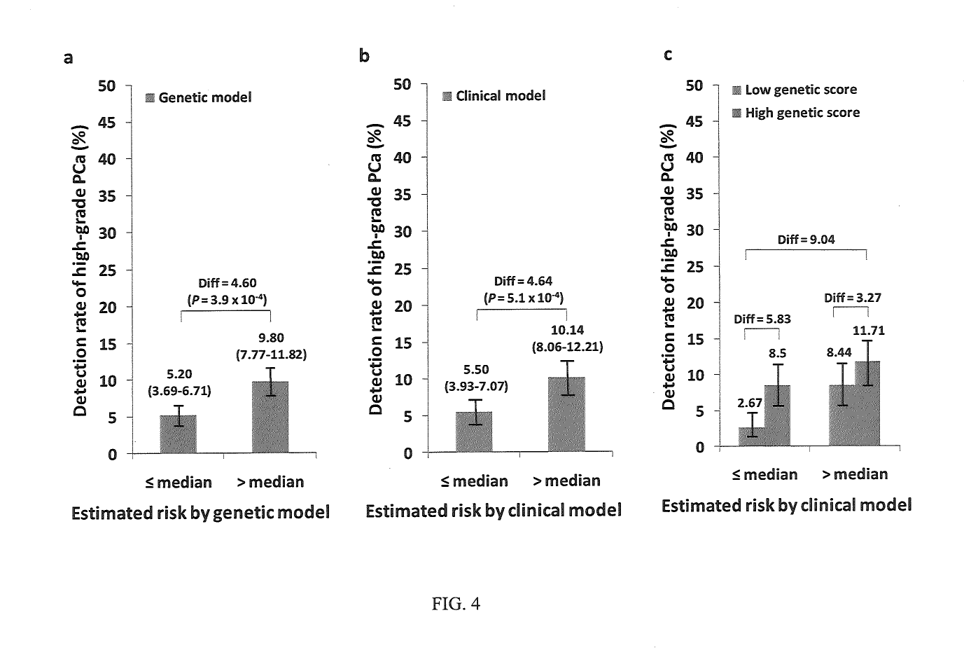

To preliminarily evaluate the performance of genetic score and clinical parameters in distinguishing risk for high-grade PCa, the detection rate of high-grade PCa among men with various estimated risk was compared under these two models. Among the 410 men who were diagnosed with PCa, 124 (30%) had high-grade PCa (Gleason grade≧7). Higher detection rates were observed among men with higher estimated risk compared to those with lower risk under the genetic model (FIG. 4a), the best clinical model (FIG. 4b), and the combination of both models (FIG. 4c).

In this study, it was found that the genetic score is a significant predictor of positive prostate biopsy and that this association is independent of known clinical parameters and family history (P=3.58×10⁻⁸). Considering that the genetic score was based on all 33 a priori established PCa risk-associated SNPs and using OR estimates obtained from external study populations, these results provide the highest level of independent evidence of the validity of these genetic markers to predict an individual's risk for PCa. In addition, through a direct comparison of the predictive performance (AUC) of genetic markers and existing clinical variables in the same study population, it was shown that the genetic score outperformed any other individual clinical parameter, including PSA, for PCa risk prediction. More importantly, the genetic score improved the AUC when added to a model including the best, existing clinical variables.

The strongest support for the predictive performance of genetic markers and added value of genetic markers to the existing clinical variables in this population is demonstrated by the measurement of detection rate of PCa. The ˜10% difference in detection rate of PCa between higher or lower genetic score and ˜20% difference between the two extreme groups (men with lower clinical risk and lower genetic score, or higher clinical risk and higher genetic score) may be clinically significant. This improvement is worth noting considering that few other biomarkers in the past several decades, be they proteins or genetic markers, have reached such a level. It is also important to note that detection rate, as a measurement of predictive performance, can be easily understood and interpreted by physicians and patients. This is in contrast to AUC, another commonly used measurement of predictive performance, where the value is not directly related to meaningful clinical measurements.

There are fundamental differences between the genetic score and clinical variables. An advantage of clinical variables is that they directly assess parameters that are associated with the development of the disease. On the other hand, the genetic score assesses the likelihood of developing disease and thus is time-independent. It can be assessed at any stage, before or after the development of disease. The high stability of DNA molecules as well as accurate and low cost genotyping of genetic markers also facilitates their clinical implementation. Some potential applications of genetic markers may include the identification of high risk men at a younger age for PCa screening and chemoprevention, as well as supplementation of the clinical variables to determine the need for biopsy or, as in this study, the need for repeat biopsy.

Results from this study not only add further support for the utility of genetic markers in predicting PCa risk but also provide new information that is urgently needed for the management of the ˜750,000 American men yearly who have a negative prostate biopsy. Currently, PSA levels and free/total PSA ratio are the primary predictors used to determine the need and interval for repeat prostate biopsy. Their ability to predict PCa is unsatisfactory, with published AUCs in the 0.60-0.75 range. The predictive performance of PSA was even lower in this study, with an AUC of 0.54 for total PSA or free/total PSA ratio. The lower AUC estimate in this study may be due to the repeat biopsy population or the fewer PSA-driven biopsies (less than 7% PCa were detected by protocol-independent biopsies). In addition, the AUCs reported in this study were based on testing samples of four-fold cross-validation, which minimizes the upward bias due to model over-fitting. Regardless of the different estimates of AUC from different studies, the generally low AUC in all of the studies points to the need for additional markers to better guide indications for repeat biopsy and determine the timing of follow-up. To this end, this study has successfully demonstrated that a genetic score based on PCa risk-associated SNPs may be one of these much needed markers.

This study validated the association of a genetic score based on 33 SNPs with PCa risk in the context of a prospective clinical trial, and for the first time, demonstrated the added value of genetic markers to the existing clinical variables for PCa prediction, The improvement of genetic markers in predicting PCa, albeit moderate, is much needed for urologists and their patients to determine the need for biopsy, and in particular repeat biopsy, for PCa detection.

### Example 3

### Additional Description and Data

Background of the Problem that is Addressed.

Prostate cancer (PCa) is the most common solid organ malignancy affecting American men and the second leading cause of cancer related death. There are at least two major problems in diagnosing and preventing PCa: 1) it is difficult to predict men at elevated risk for PCa, and 2) it is difficult to predict outcome of prostate biopsy.

Recently, 33 PCa risk-associated single nucleotide polymorphisms (SNPs) have been identified. This study was conducted to assess the ability of these 33 inherited PCa risk-associated genetic markers to address the problems listed above.

Brief Summary of the Invention.

Using clinical data and DNA samples from the

REduction by DUtasteride of prostate Cancer Events (REDUCE) trial, results were obtained that may have broad clinical utility:
a) Genetic score based on a panel of 33 PCa risk-associated SNPs (PCS33) can predict an individual's risk for PCa.
b) Genetic score based on PCS33 can supplement current clinical variables (PSA, prostate volume, age, and family history) to better determine the clinical decision to pursue prostate biopsy (or repeat prostate biopsy) for detection of PCa.

Among the 1,654 men of European descent who had an initial negative biopsy for PCa and who consented to genetic study in the placebo arm of the REDUCE trial, 410 men (25%) had a positive prostate biopsy for PCa from scheduled and for-cause biopsies over the four-year study. In a univariate analysis, men with positive biopsies had significantly higher genetic score based on PCS33 than men with negative prostate biopsy (P=4.95×10⁻⁹). After adjusting for known PCa risk-associated clinical variables such as age, free/total PSA ratio, number of cores at base biopsy, and prostate volume using multivariate logistic regression analysis, and family history, the genetic score remained significantly associated with positive prostate biopsy (P=3.58×10⁻⁸). The results from this prospective clinical trial establish the basis for the use of these genetic markers to predict an individual's risk for PCa.

The area under the receiver operating characteristic curve (AUC) was used to assess the performance of these baseline clinical variables and genetic score, individually and in combination, to predict positive prostate biopsy during the four-year follow-up. To obtain unbiased estimates of AUC, a four-fold cross validation method was used and results from testing samples were reported (Table 2). The AUC of the genetic score was highest (0.59) among individual predictors; including prostate volume (0.56), age (0.56), number of cores sampled at pre-study entry biopsy (0.55), free/total PSA ratio (0.54), total PSA (0.54), family history (0.52), and DRE (0.51). When multiple predictors were included in the model simultaneously, the AUC for commonly used predictors including age, family history, and total PSA was 0.58. The best clinical model included five baseline variables (age, family history, free/total PSA ratio, number of cores at pre-study entry biopsy, and prostate volume), with an AUC of 0.60. When the genetic score was added to this best clinical model, the AUC of the full model increased to 0.64.

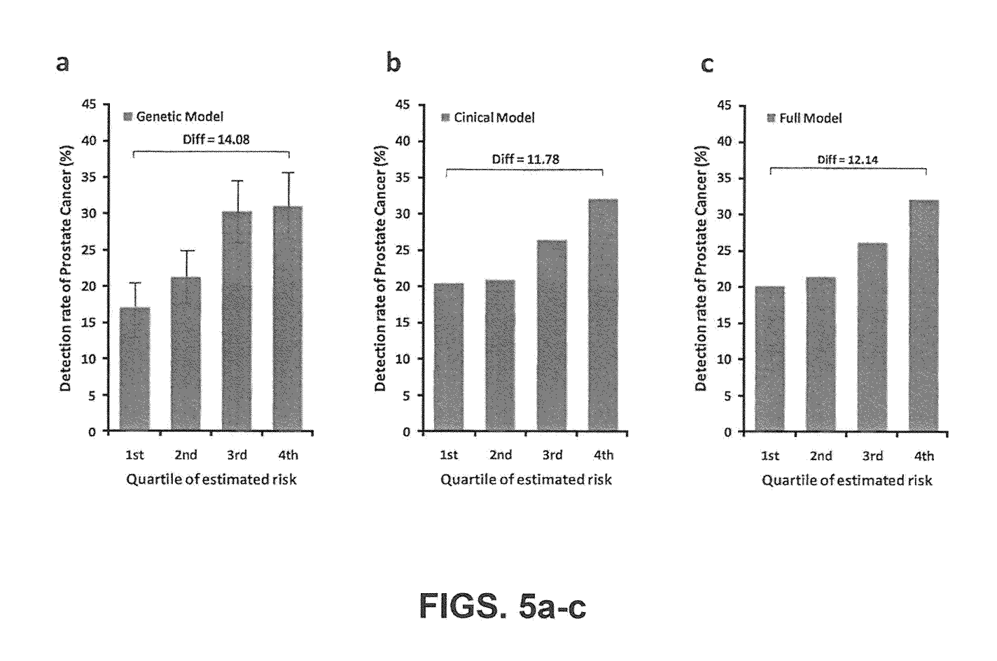

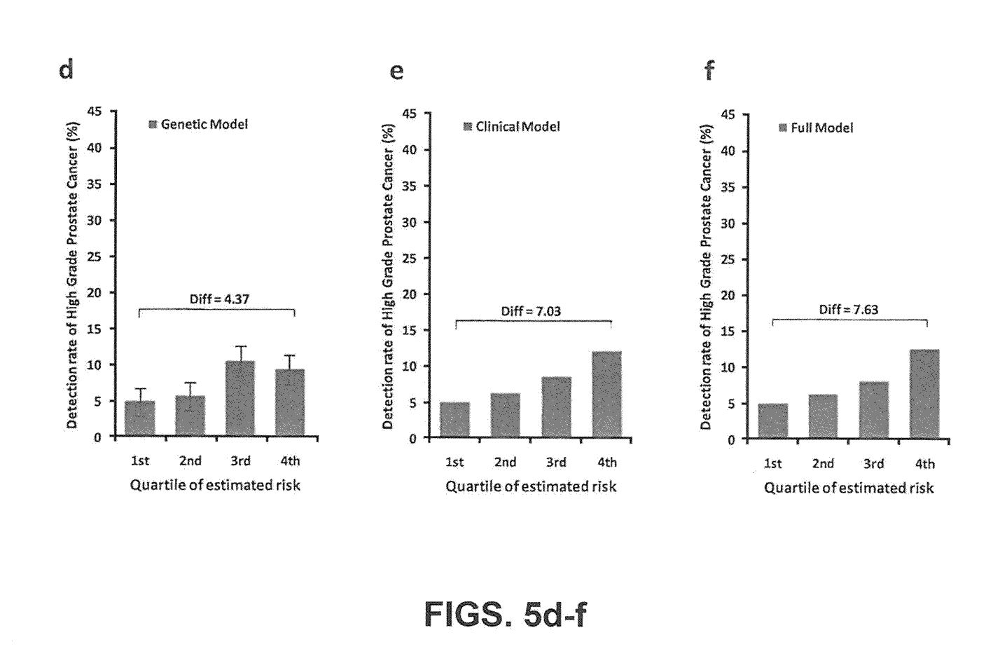

To facilitate the use and interpretation of these models in predicting positive prostate biopsy, the detection rate of PCa and high-grade PCa was calculated for the genetic score model, the best clinical model, and the full model (FIG. 5). For each model, the detection rate generally increased in men with increasingly higher estimated risk. The difference in PCa detection rate between the lowest and highest quartile was 14.08%, 11.78%, and 12.14% for the genetic score model, the best clinical model, and the full model that combined genetic score with the best clinical model, respectively (FIGS. 5a-c). The difference in high-grade PCa detection rate between the lowest and highest quartile was 4.37%, 7.03%, and 7.63% for the genetic model, the best clinical model, and the full model, respectively (FIGS. 5d-f).

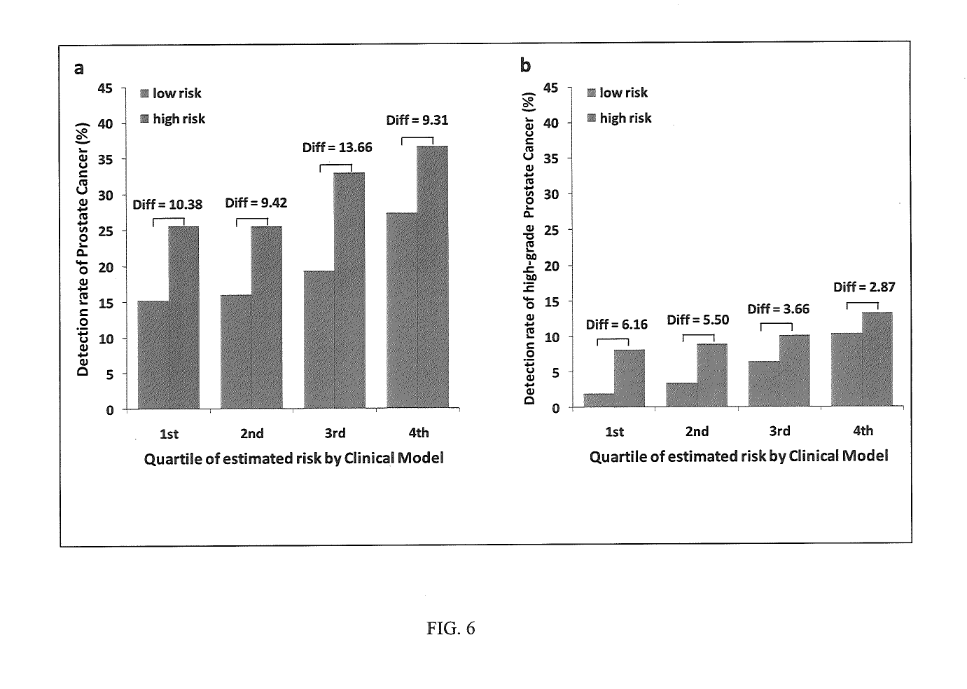

To further examine the added value of the genetic score to the existing clinical parameters in predicting positive prostate biopsy, PCa detection rates were estimated in each quartile of risk based on the best clinical model, stratified by genetic score (lower and higher half) (FIG. 6a). Within each clinical risk quartile, the detection rates differed considerably between men with lower and higher genetic scores; the difference was 10.38% in the 1st, 9.42% in the 2nd, 13.66% in the 3rd, and 9.31% in the 4th risk quartile, respectively. Comparing across the risk quartiles, men with higher genetic scores, even in the lower clinical risk quartile, had comparable or even higher PCa detection rate than men with lower genetic scores in any clinical risk quartile. Specifically, the PCa detection rate was 25.64% for men that had a higher genetic score within the lowest clinical risk quartile; this is comparable or higher than the detection rates among men that had a lower genetic score in the 2nd, 3rd, or highest clinical risk quartile (16.06%, 19.34%, and 27.34%, respectively). Similarly, genetic score was able to further differentiate the detection rate of high-grade PCa defined by the best clinical model (FIG. 6b).

Through a direct comparison of the predictive performance (AUC) of the genetic score and existing clinical variables in the same study population, it was shown that the genetic score performed better than any other individual clinical parameter, including PSA, for PCa risk prediction. More importantly, the genetic score improved the AUC of existing clinical variables. The strongest support for the added value of the genetic score to the existing clinical variables in this population is reflected by the ability of the genetic score to differentiate PCa detection rates among men in the same risk quartile defined by the best clinical model.

Prior to this study, it was not known whether reported PCa risk-associated SNPs are false positive due to PSA detection bias (i.e., these SNPs are associated with elevated PSA and not PCa risk per se, as elevated PSA leads to more prostate biopsies and in turn a greater PCa detection rate as is seen in case control studies). In addition, because many clinical variables such as PSA and DRE are commonly used to define cases and controls in case-control studies, it is difficult to assess relative predictive performance of genetic markers and clinical variables such as PSA, and more importantly whether genetic markers considerably improve the ability of existing clinical parameters to predict for PCa.

The placebo arm of the REDUCE study, a large randomized clinical trial, provided a unique opportunity to answer these questions. All men in the study had a negative biopsy at baseline and were followed-up for four years, with scheduled not-for-cause prostate biopsies at years 2 and 4. In addition, because it is a clinical trial, a number of clinical variables, such as free/total PSA ratio and prostate volume were measured at baseline using a standardized protocol. These findings establish the clinical validity of these PCa risk-associated SNPs and the value they add to existing clinical variables for the prediction of PCa risk in a large prospective clinical trial.

### Example 4

### Analysis of Randomly Selected Subsets of the 33 SNPs of Table 1

Calculations as described herein were performed on 10 and 15 randomly selected SNPs (Table 6) that are subsets of the 33 SNPs of Table 1 and this random sampling was repeated five times. The genetic scores (CRRs) calculated from these subsets is equivalent or better that the family history for detecting prostate cancer risk measured by AUC (Table 5).

### Example 5

### Charts for Prostate Biopsy

Summary.

Despite the fact that moderately elevated total prostate-specific antigen (tPSA) level (2.5-10 ng/mL) is a poor predictor of prostate cancer (PCa) and recent findings that several relevant biomarkers have a better predictive performance of PCa than tPSA, elevated tPSA levels remain the primary indication for prostate biopsy for detection of PCa in clinics. While many factors may contribute to this dilemma, a primary factor may be the lack of an informative tool to illustrate the added value of novel biomarkers over tPSA. To overcome this barrier, charts were developed for determining a prostate cancer detection rate based on genetic score (GRS), a tool that visually illustrates the added value of genetic score derived from PCa risk-associated SNPs over existing clinical predictors, age and tPSA, and can assist prostate biopsy decision making. These charts can be extended to include additional established biomarkers such as serum [−2]proPSA (p2PSA), from which a prostate health index (PHI) is derived and urine PCA3. The chart can also provide prostate cancer detection rates based on tPSA levels in combination with GRS and PHI. On the basis of information provided in these charts, a subject can make a better informed decision regarding whether to undergo an initial prostate biopsy or repeat biopsy. The simplicity and informativeness of these charts facilitate wide adoption of these biomarkers, with the ultimate goal of significantly improving the detection rate of PCa, especially for aggressive PCa, while reducing the number of unnecessary prostate biopsies.

Limited Predictive Value of PCa for Moderately Elevated PSA Levels.

Elevated tPSA level is currently the primary indication for prostate biopsy. While considerably elevated tPSA level (>10 ng/mL) is an excellent predictor for PCa, the vast majority of these patients have moderately elevated tPSA levels (2.5-10 ng/mL), which is known to be a poor predictor of PCa. The best evidence came from the Prostate Cancer Prevention Trial (PCPT), where all men had a trial-mandated biopsy regardless of PSA levels. Among 5,519 subjects from the placebo group, the overall PCa detection rate was 22%, and the rate was only slightly higher in patients with tPSA levels>2 ng/mL (34%) than those with PSA levels<=2 ng/mL (15%).

Biomarkers for Predicting PCa.

Several biomarkers have been consistently demonstrated to have a better predictive performance for PCa than moderately elevated tPSA. For example, a genetic risk score (GRS) derived from 33 PCa risk-associated SNPs has been shown to be a significantly stronger predictor of PCa than tPSA, in a for-cause prostate biopsy cohort in Stockholm, Sweden and in a repeat prostate biopsy cohort in the REDUCE® trial. p2PSA and its derivative Prostate Health Index (PHI) have been consistently shown to be a better predictor of PCa than tPSA alone in initial and repeat biopsy. An FDA approved kit for measurement of p2PSA and PHI was recently approved. Similarly, a non-coding RNA, prostate cancer antigen 3 (PCA3), has also been consistently demonstrated to be a superior predictor of PCa than tPSA in initial and repeat biopsy. Urine PCA3 was also approved by the FDA to help determine the need for repeat prostate biopsies. In addition, there is some evidence that both p2PSA and PCA3 may perform better to identify high-grade PCa.

Barriers for Adoption of Novel Biomarkers.

Despite overwhelming evidence that these biomarkers have better predictive performance for PCa than moderately elevated tPSA, tPSA remains the primary and only indication for prostate biopsy in most clinics. This practice and the poor predictive performance of tPSA (both specificity and sensitivity) contribute to the problem of over-biopsy and false negatives. Multiple factors may contribute to the low adoption of the use of these biomarkers. However, primary factors may be a lack of an informative tool to illustrate the added value of these biomarkers over tPSA.

Chart for Prostate Biopsy.

The Chart for Prostate Biopsy is a simple and informative tool that clearly illustrates the need for additional biomarkers to improve decision-making regarding prostate biopsy. The average detection rate of PCa is indicated for patients in each of these groups (vertical black bar). In addition, in each of these groups, the average detection rate of PCa is also indicated for patients with low (<0.5, triangle), intermediate (0.5-1.5, square), and high (>1.5, diamond) genetic risk score, as estimated from 33 PCa risk-associated SNPs (FIG. 7). The chart can be informative for both urologists and patients, by showing the added value of genetic score in estimating an individual's PCa detection rate.

As one nonlimiting example, subjects A, B and C are all in the group of 55-59 year olds with tPSA of 4.0-9.9, and their expected PCa detection rate is the same at 41% without genetic risk information. After a genetic test of the 33 PCa risk-associated SNPs, subjects A, B and C each found out their genetic risk score was 0.4, 1.2, and 2.1, respectively. Therefore, their expected PCa detection rate is 20%, 39%, and 54%, respectively. Because subject A's expected detection rate was close to 19%, the expected PCa detection rate of men with PSA<2.5 in his group, he and his urologist decided to forgo prostate biopsy at this time. For subject B, he and his urologist were undecided and planned to follow-up his tPSA. Subject C decided to have a prostate biopsy because his expected PCa detection rate was more than 50%.

This chart is much easier to use compared to nomograms where urologists have to draw complicated lines. Furthermore, the chart can be extended to include p2PSA and the calculated prostate health index, PHI), PCA3, and other predictors to further refine their expected PCa detection rare. This method can also be used to calculate the detection rate of high-grade PCa.

Thus, this invention demonstrates the potential to develop the first user-friendly tool for prostate biopsy decision-making. The simplicity and informativeness of the chart of this invention may facilitate wide adoption of these biomarkers to significantly improve the detection rate of PCa, especially aggressive PCa, while reducing the number of unnecessary prostate biopsies.

### Example 6

### Further Description of Charts for Prostate Biopsy

Elevated serum prostate-specific antigen (PSA) level is the primary indication for prostate biopsy for detection of prostate cancer (PCa) in the modern era. The detection rate of PCa from biopsy is typically below 30%, especially among patients with PSA levels at 4-10 ng/mL. In the past several years, additional biomarkers, such as Prostate Health Index (PHI), PCA3, and genetic risk score (GRS) derived from multiple PCa risk-associated SNPs have been shown to provide added value to PSA in discriminating prostate biopsy outcomes.

To overcome the low specificity of PSA for predicting PCa and reduce over-biopsy, extensive efforts have been devoted to develop other biomarkers. A PSA-related biomarker is a truncated PSA isoform, [−2]proPSA (p2PSA). A systematic review and meta-analysis demonstrated that serum p2PSA has greater accuracy than tPSA or fPSA in detecting PCa in men with a tPSA between 2 and 10 ng ml. Furthermore, a prostate health index (PHI), derived from a combination of p2PSA, tPSA and fPSA, has been shown to be a better predictor of PCa. PHI tests for men 50 years and older with a tPSA value between 4 and 10 ng ml-1 and a digital rectal exam (DRE) with no suspicion of cancer by Beckman Coulter Inc have been approved by the European Medicines Agency and the United States Food and Drug Administration.

However, the adoption rate of these novel biomarkers in clinics is low, largely due to poor understanding of the added value of novel biomarkers. To address this matter, a chart was developed to visually present 1) expected detection rates of PCa from biopsy with respect to PSA levels, and more importantly, 2) a range of PCa detection rates at the same PSA levels when novel biomarkers are considered. This chart, called the Xu's chart for prostate biopsy, is not a formal risk prediction model; rather, a simple visual tool for urologists to communicate with their patients an initial evaluation of PCa detection rate based on their PSA levels and a possible recommendation for additional biomarkers. A more comprehensive evaluation of PCa risk using existing risk assessment tools such as nomograms can be followed once additional biomarkers are measured.

Prostate cancer (PCa) is the second most frequently diagnosed cancer and the sixth leading cause of cancer-related death in men, with an estimated 914,000 new cases and 258,000 deaths per year globally in 2008. PCa incidence rate differs widely among countries and regions, possibly due to differences in the adoption rate of prostate-specific antigen (PSA) screening for PCa, as well as inherited risk and environmental exposures such as diet. The trend of PCa incidence in the last several decades also differed considerably among various countries and regions. In the United States, the incidence rate increased sharply in the early to mid-1990s with the introduction of PSA screening for PCa, and declined since. In Shanghai, China, the age-adjusted incidence rate of PCa increased from 2.3 per 100,000 during 1988-1992 to 6.9 per 100,000 during 1998 to 2002, and reached 16.0 per 100,000 in 2007. The 7-fold increase of PCa incidence in Shanghai coincided with the gradual introduction of PSA screening for PCa during that period of time.

In developed countries and many developing countries where modern medical services are readily accessible, most PCa is diagnosed from prostate biopsy among asymptomatic men with elevated PSA levels through a systematic PSA screening or incidental PSA tests. While PCa detection rate is typically over 50% among patients with considerably elevated PSA levels (for example, >10 ng/mL), its detection rate is generally low, especially among those with moderately elevated PSA levels (4-10 ng/mL). For example, the overall PCa detection rates were only 33.0% among 25,733 patients who underwent prostate biopsy in 10 biopsy cohorts from the Prostate Biopsy Collaborative Group (Table 7). The PCa detection rate was 25.2%, 33.8%, and 56.3% among patients with PSA<4 ng/mL, 4-10 ng/mL, and >10 ng/mL, respectively. Similar results were found in Chinese men. In a hospital-based study of 667 consecutive patients who underwent prostate biopsy at two tertiary hospitals in Shanghai, China between 2011 and 2012, the PCa detection rate was 39.0% in the entire cohort, and was 17.7% and 52.3% in patients with PSA at 4-10 ng/mL and >10 ng/mL, respectively.

The overall low detection rate of PCa from biopsy may be attributed to the fact that PSA is prostate specific, but not PCa specific. Many non-cancer factors such as enlarged prostate and inflammation in the prostate may also lead to elevated PSA levels. Therefore, a decision of prostate biopsy based on PSA levels alone may lead to many unnecessary biopsies. Prostate biopsy is an invasive procedure and is often associated with potential harms. It is estimated that one third of men who have prostate biopsy experience pain, fever, bleeding, infection, transient urinary difficulties, or other issues requiring clinician follow-up, and approximately 1% require hospitalization.

To overcome the low specificity of PSA for predicting PCa and reduce over-biopsy, extensive efforts have been devoted to develop other biomarkers. One such biomarker is serum free PSA (fPSA), the form of PSA that is unbounded by protein. It has been shown that men with PCa have a lower % fPSA [proportion of fPSA in total PSA (tPSA)] than those without PCa. Several studies demonstrated that a cutoff of 14-28% could reduce unnecessary biopsies by 19-64% while maintaining a sensitivity of 71-100%.

Another related biomarker is a truncated PSA isoform, [−2]proPSA (p2PSA). A systematic review and meta-analysis demonstrated that serum p2PSA has greater accuracy than tPSA or fPSA in detecting PCa in men with a tPSA between 2 and 10 ng/mL. Furthermore, a prostate health index (PHI), derived from a combination of p2PSA, tPSA, and fPSA, has been shown to be a better predictor of PCa. A PHI test for men 50 years and older with a tPSA value between 4-10 ng/mL and a digital rectal exam (DRE) with no suspicion of cancer by Beckman Coulter Inc. has been approved by the European Medicines Agency (EMA) and the United States Food and Drug Administration (FDA).

Several urine biomarkers for PCa have also been developed. The prostate cancer antigen 3 (PCA3) gene is over-expressed in PCa tissue compared with adjacent BPH or normal prostate tissue. In addition, PCA3 expression is not detectable in non-prostatic normal tissues and tumors, suggesting that PCA3 is PCa specific. A quantitative urinary assay for PCA3 messenger RNA (mRNA) has been developed, with an area under the receiver operating characteristic curve (AUC) of ˜0.75 in discriminating prostate biopsy outcomes. Its predictive performance was later confirmed in multiple studies, with an AUC from 0.69 to 0.75 for discriminating PCa and high-grade PCa. A urine PCA3 test for considering a repeat biopsy in men 50 years of age or older who have had one or more previous negative prostate biopsies by Hologic Gen-Probe has been approved by the EMA and FDA.

Similar to the PCA3 test, a urine biomarker of mRNA for the fusion gene TMPRESS2-ERG has also been developed. The fusion gene is commonly found in prostate tumors. Its AUC for discriminating PCa from non-PCa in prostate biopsy was ˜0.77. Results from a multi-center study suggested that TMPRSS2-ERG had independent additional predictive value when compared to PCA3 for biopsy outcomes.

Another type of PCa biomarkers is inherited genetic markers. Genetic susceptibility to PCa is well established from twin studies and family studies. Specifically, more than 70 PCa risk-associated SNPs have been identified using genome-wide association studies (GWAS) in the last several years. These SNPs have been consistently associated with PCa risk in multiple study populations. A genetic risk score (GRS) derived from a combination of these risk-associated SNPs can be used to predict inherited risk for PCa. The predictive performance of GRS derived from the first 33 PCa risk-associated SNPs was recently confirmed within the context of a clinical trial [REduction by DUtasteride of prostate Cancer Events (REDUCE)]. It was demonstrated that they perform significantly better (AUC=0.59) than many existing clinical parameters to predict positive biopsy during the four-year trial, including tPSA (AUC=0.53), % fPSA (AUC=0.54), and family history (AUC=0.52). The increased performance of GRS over family history, another commonly used measurement for inherited risk, was supported from multiple study populations. In addition to subjects from European descent, the added value of GRS derived from multiple PCa risk-associated SNPs to PSA in discriminating biopsy outcomes was also demonstrated in the Chinese population, especially among patients with moderately elevated PSA levels.

Despite extensive evidence for the added value of these novel biomarkers to PSA and that some of these biomarkers are approved by FDA, their adoption in clinics for assisting PSA in determining the need for biopsy is low. Many factors may contribute to this dilemma, including the fact that these biomarkers have not been adopted in various clinical guidelines and the costs for measuring these biomarkers. For example, no statement about PCA3 and p2PSA is mentioned in the National Comprehensive Cancer Network (NCCN) and American Urological Association (AUA). In the European Union (EU) guidelines (2013), it states “main current indication of the PCA3 urine test may be used to determine whether a man needs a repeat biopsy after an initially negative biopsy outcome, but its cost-effectiveness remains to be shown.” Another major factor is a lack of appreciation of the added value of these biomarkers to the existing clinical predictors in a clinical and practical sense. This is in part because most statistical measurements in the literature for assessing the performance of biomarkers do not have direct clinical meaning. For example, AUC is an excellent and widely used statistical measurement for assessing discriminative performance of a test. However, it does not directly convey clinical information to urologists and patients regarding their biopsy outcomes. Therefore, other approaches are urgently needed to translate these biomarkers from research into clinics.

The present invention provides a chart that shows simple measurement, expected PCa detection rate, to improve appreciation of the added value of novel biomarkers to PSA in making a decision for prostate biopsy. The chart is used to visually present 1) expected detection rates of PCa from biopsy with respect to PSA levels, and more importantly, 2) a range of PCa detection rate at the same PSA levels when a biomarker is considered. This chart, called the Xu's chart for prostate biopsy, offers a simple and informative tool for urologists to discuss expected biopsy outcomes and the added value of additional biomarkers with their patients prior to biopsy. It provides more clinically meaningful information to urologists and patients than commonly used measurements such as AUC. Described below is an example of the chart of this invention, using GRS as an example, to demonstrate its effect in improving appreciation of the added value of biomarkers.

As described above, more than 70 PCa risk-associated SNPs have been consistently discovered from GWAS. Although GRS derived from these risk-associated SNPs has been consistently shown to be a significant and independent predictor of PCa from biopsy, few clinicians use GRS in clinics to assess their patients' genetic risk for PCa. In contrast, clinicians and patients rely greatly on family history to achieve this goal, even though family history is less objective and performs worse than GRS. Better understanding of how GRS can add value to PSA in making a decision of prostate biopsy may promote its use in clinics.

As described herein, 33 PCa risk-associated SNPs were genotyped in subjects from a population-based biopsy cohort from Sweden and the placebo arm of REDUCE. We then A GRS was calculated for each individual based on their genotypes at these 33 SNPs, the odds ratio (OR) of these SNPs derived from an external meta-analysis, and the allele frequency of these SNPs in the CEU (Caucasian) population. Because GRS is relative to a general population, a GRS of 1.0 indicates an average inherited risk for PCa in the general population. Consequently, each subject can be classified as low-, intermediate-, and high-inherited risk groups, if their GRS is <0.5, 0.5-1.5, and >1.5, respectively.

The average PCa detection rates from biopsy and their 95% confidence interval (95% CI) for men with different PSA levels (4-6.9, 7.0-9.9, and >10 ng/mL) are presented in Table 8. In addition, in each of these PSA groups, the average PCa detection rates for subjects with low-, intermediate-, and high-inherited risk for PCa are also presented. Finally, these PCa detection rates and the 95% CI are plotted in a chart for a visual presentation (FIG. 7).

Several pieces of information are clearly noticeable from the chart. First, each patient can easily find out his expected PCa detection rate based on his PSA level prior to biopsy. For example, if a patient's PSA is at 4-6.9 ng/mL, he will find he has a 43.1% chance to be diagnosed with PCa from a biopsy. Based on the expected detection rate and other factors as well as considering potential benefits and harms, his urologist may or may not recommend a biopsy at this time. On the other hand, if a patient's PSA is >=10 ng/mL, he will find he has a 75.1% chance to be diagnosed with PCa. Therefore, his urologist will most likely recommend a biopsy. Second, for patients whose PSA levels are in the grey zone (4-9.9 ng/mL), they will notice that they would have much more information regarding their expected PCa detection rate if they know their genetic risk for PCa. For example, for men whose PSA levels are between 4-6.9 ng/mL, the expected PCa detection rate would be as low as 27.9% if their GRS is <0.5 (12% of men in the group) or as high as 54.6% if their GRS is >1.5 (25% of men in the group). As a result, it is easier for urologists and patients to appreciate the added value of genetic risk and opt for measuring this biomarker. The additional information from GRS may offer a better assessment of biopsy outcomes and therefore reduce unnecessary biopsy for many patients while improving the detection rate of PCa in a subset of patients.

A Xu's chart for biopsy was also developed for Chinese men (FIG. 8 and Table 9). The data were based on a biopsy cohort from two tertiary hospitals in Shanghai, China. A GRS was calculated for each patient based on the 13 strongest PCa risk-associated SNPs in Chinese men (Table 10). Again, two points are clearly conveyed by the chart. First, each patient can easily find out his PCa detection rate based on his PSA level; 17.7%, 35.3%, or 70.7% if his PSA level is at 4-9.9, 10-19.9, or >=20 ng/mL, respectively. It is interesting to note that the detection rate of PCa in Chinese men is considerably lower than Caucasian men, and the PSA level grey zone in Chinese men is not 4-9.9, but 10.1-19.9 ng/mL. Second, the added value of GRS in estimating PCa detection is more prominent for patients in the grey zone PSA levels. The expected detection rate of PCa based on PSA alone is moderate for this group (35.3%). However, the rate would be as low as 7.7% if the patient has a low genetic risk (GRS<0.5) or as high as 47.6% if they have a high genetic risk (GRS>1.5). In contrast, the added value of GRS is limited for patients with relatively low PSA (4-9.9 ng/mL) or very high PSA levels (>20 n/mL). Although the PCa detection rate ranges from 7.1%-24.6% between low and high GRS for patients with PSA at 4-9.9 ng/mL, they are all relatively low to consider for a biopsy. Similarly, for patients with PSA>20 ng/mL, even though the detection rate ranges from 55.0% to 81.8% between low and high GRS, they are all high enough to warrant a biopsy.

The primary purpose of the Xu's chart for prostate biopsy is to provide a simple and practical tool for urologists to discuss with their patients prior to biopsy. If this chart is available at each urological clinic, urologists can use it to explain to their patients what they can expect from biopsy based on PSA information alone or if additional information from other biomarkers are available. This would promote the uptake of novel biomarkers in clinics for a better assessment of PCa risk using more comprehensive risk assessment tools. Together with a discussion of potential benefits and harms of biopsy, urologists and patients can make an informed decision regarding the need for a prostate biopsy.

The key advantage of the chart is that the information it conveys (PCa detection rate) addresses a primary concern of patients and therefore can be easily understood. It is important to note that the chart is not a formal risk prediction model; rather, it is a simple tool for urologists to communicate initial evaluations and recommendations for additional biomarkers based on their individual PSA levels. It differs from other well-established statistical measurements for discriminating biopsy outcomes such as AUC, Integrated Discrimination Improvement (IDI), Net Reclassification Index (NRI), and Decision Curve Analysis (DCA). These measurements capture the overall discriminative performance of a test at a population level but do not directly convey clinically meaningful information to individual patients. It is also important to note that the chart does not intend to compete with but complements sophisticated risk prediction tools such as such as nomograms, the Prostate Cancer Prevention Trial (PCPT) Risk calculator, and the Cancer of the Prostate Risk Assessment (CAPRA) score. It serves as the first step to preliminarily assess patients' risk for PCa based on PSA levels and to encourage a subset of patients to obtain additional biomarkers for a comprehensive evaluation of PCa risk using these tools. This is a practical and important issue in a busy clinic, especially in China where urologists typically see several dozens of patients in a day.

Several modifications to the chart can be considered. First, with a larger sample size, the chart can include PSA levels at different age groups. Second, in addition to plotting the detection rate of any PCa, it is more important to plot PCa detection rate of high-grade PCa. Third, the chart can be extended to other novel biomarkers such as PHI (FIGS. 9 and 10), PCA3, and TMPRESS2-ERG. It is expected that the detection rate of PCa and high-grade PCa could be further differentiated based on a combination of these biomarkers.

The foregoing is illustrative of the present invention, and is not to be construed as limiting thereof. The invention is defined by the claims provided herein, with equivalents of the claims to be included therein.

All publications, patent applications, patents, patent publications, sequences identified by GenBank® Database accession numbers and/or SNP accession numbers, and other references cited herein are incorporated by reference in their entireties for the teachings relevant to the sentence and/or paragraph in which the reference is presented.

TABLE 1
SNPs associated with PCa and their odds ratio from a meta-analysis

| | | | | Known | m/M* | Risk | OR |
|---|---|---|---|---|---|---|---|
| CHR | SNPs | Note | BP-build36 | genes | allele | allele | (95% CI) |
| 2 | rs1465618 | 2p21 | 43,407,453 | THADA | A/G | A | 1.15 (1.04-1.26) |
| 2 | rs721048 | 2p15 | 62,985,235 | EHBP1 | A/G | A | 1.16 (1.11-1.22) |
| 2 | rs12621278 | 2q31.1 | 173,019,799 | ITGA6 | G/A | A | 1.35 (1.27-1.44) |
| 3 | rs2660753 | 3p12 | 87,193,364 | | T/C | T | 1.24 (1.04-1.48) |
| 3 | rs10934853 | 3q21.3 | 129,521,063 | | A/C | A | 1.12 (1.06-1.18) |
| 4 | rs17021918 | 4q22.3 | 95,781,900 | PDLIM5 | T/C | C | 1.14 (1.10-1.18) |
| 4 | rs7679673 | 4q24 | 106,280,983 | TET2 | A/C | C | 1.13 (1.10-1.17) |
| 6 | rs9364554 | 6q25 | 160,753,654 | | T/C | T | 1.17 (1.06-1.29) |
| 7 | rs10486567 | 7p15 | 27,943,088 | JAZF1 | A/G | G | 1.16 (1.10-1.23) |
| 7 | rs6465657 | 7q21 | 97,654,263 | LMTK2 | T/C | C | 1.14 (1.05-1.23) |
| 8 | rs2928679 | 8p21.2 | 23,494,920 | NKX3.1 | A/G | A | 1.13 (1.02-1.25) |
| 8 | rs1512268 | 8p21.2 | 23,582,408 | NKX3.1 | T/C | T | 1.17 (1.14-1.21) |
| 8 | rs10086908 | 8q24 (5) | 128,081,119 | | C/T | T | 1.13 (1.09-1.18) |
| 8 | rs16901979 | 8q24 (2) | 128,194,098 | | A/C | A | 1.80 (1.57-2.06) |
| 8 | rs16902094 | 8q24.21 | 128,389,528 | | N/A | G | 1.20 (1.12-1.30) |
| 8 | rs620861 | 8q24 (4) | 128,404,855 | | A/G | G | 1.16 (1.11-1.20) |
| 8 | rs6983267 | 8q24 (3) | 128,482,487 | | G/T | G | 1.20 (1.14-1.26) |
| 8 | rs1447295 | 8q24 (1) | 128,554,220 | | A/C | A | 1.47 (1.33-1.62) |
| 9 | rs1571801 | 9q33 | 123,467,194 | | G/A | A | 1.17 (0.95-1.45) |
| 10 | rs10993994 | 10q11 | 51,219,502 | MSMB | T/C | T | 1.25 (1.12-1.40) |
| 10 | rs4962416 | 10q26 | 126,686,862 | CTBP2 | C/T | C | 1.15 (1.04-1.27) |
| 11 | rs7127900 | 11P15.5 | 2,190,150 | IGF2, IGF2AS, INS, TH | G/A | A | 1.25 (1.20-1.30) |
| 11 | rs12418451 | 11q13 (2) | 68,691,995 | AL137479, BC043531 | A/G | A | 1.16 (1.09-1.23) |
| 11 | rs10896449 | 11q13 (1) | 68,751,243 | | A/G | G | 1.16 (1.11-1.22) |
| 17 | rs11649743 | 17q12 (2) | 33,149,092 | | A/G | G | 1.16 (1.11-1.22) |
| 17 | rs4430796 | 17q12 (1) | 33,172,153 | TCF2 | A/G | A | 1.22 (1.17-1.26) |
| 17 | rs1859962 | 17q24.3 | 66,620,348 | | G/T | G | 1.20 (1.13-1.27) |
| 19 | rs8102476 | 19q13.2 | 43,427,453 | | T/C | C | 1.12 (1.08-1.15) |
| 19 | rs887391 | 19q13 | 46,677,464 | 10 Mb to KLK3 | C/T | T | 1.14 (1.08-1.20) |
| 19 | rs2735839 | 19q13 (KLK3) | 56,056,435 | KLK3 | A/G | G | 1.30 (1.11-1.51) |
| 22 | rs9623117 | 22q13 | 38,782,065 | | C/T | C | 1.13 (1.05-1.22) |
| 22 | rs5759167 | 22q13.2 | 41,830,156 | TTLL1, BIK, MCAT, PACSIN2 | T/G | G | 1.18 (1.14-1.21) |
| 23 | rs5945619 | Xp11 | 51,258,412 | NUDT10, NUDT11, LOC340602 | C/T | C | 1.27 (1.12-1.43) |

*m = minor allele, M = major allele.

TABLE 2
Clinical and genetic predictors of prostate
cancer and high-grade prostate cancer

| | Testing AUC from four-fold cross validation | |
|---|---|---|
| | Any | High-grade |
| | prostate | prostate |
| Variables and models | cancer | cancer |
| Individual variables | | |
| at baseline | | |
| Age at baseline (Age) | 0.56 | 0.61 |
| Digital rectal examination | 0.51 | 0.50 |
| at baseline (DRE) | | |
| Total PSA levels at baseline | 0.54 | 0.59 |
| Free/total PSA ratio at | 0.54 | 0.57 |
| baseline (f/t PSA) | | |
| Prostate volume at | 0.56 | 0.59 |
| baseline (PV) | | |
| Number of cores sampled | 0.55 | 0.58 |
| at base biopsy | | |
| (No. of cores) | | |
| Family history at baseline | 0.53 | 0.54 |
| (FH) | | |
| Genetic score based on 33 | 0.59 | 0.57 |
| PCa risk SNPs | | |
| (Genetic score) | | |
| Combined variables | | |
| Age + FH + total PSA | 0.58 | 0.65 |
| Age + FH + f/t PSA | 0.59 | 0.65 |
| Age + FH + DRE + f/t | 0.59 | 0.65 |
| PSA | | |
| Age + FH + f/t PSA + | 0.60 | 0.67 |
| PV + No. of cores | | |
| Age + FH + f/t PSA + | 0.64 | 0.67 |
| PV + No. of cores + | | |
| Genetic score | | |

High-grade prostate cancer is defined as Gleason grade 7 or higher

TABLE 3
Baseline clinical, demographic, and genetic score of the subjects in the study

| | All subjects | | | | | Subjects with positive Biopsies | | | | |
|---|---|---|---|---|---|---|---|---|---|---|
| | Positive | | Negative | | P- | Gleason | | Gleason | | P- |
| Variables | Biopsies | | Biopsies | | values | grade ≦6 | | grade ≧7 | | values |
| Number of subjects | 410 | | 1244 | | | 286 | | 124 | | |
| Age at baseline | | | | | | | | | | |
| Mean (SD), years | 63.52 | (5.99) | 62.22 | (6.01) | 0.0001 | 63.01 | (6.02) | 64.72 | (5.75) | 0.008 |
| Range | 50-76 | | 49-76 | | | 50-76 | | 52-75 | | |
| # (%) with positive family history at baseline | 68 | (17%) | 146 | (12%) | 0.01 | 44 | (15%) | 24 | (19%) | 0.32 |
| # (%) with positive DRE ^{†} at baseline | 20 | (5%) | 47 | (4%) | 0.33 | 15 | (5%) | 5 | (4%) | 0.60 |
| Total PSA levels at baseline | | | | | | | | | | |
| Mean (SD), mL | 5.78 | (1.37) | 5.52 | (1.40) | 0.01 | 5.62 | (1.37) | 6.16 | (1.36) | 0.008 |
| Range, mL | 2.5-10.2 | | 1.8-14.2 | | | 2.5-10.2 | | 2.7-10 | | |
| Free/total PSA ratio at baseline | 0.16 | (0.06) | 0.17 | (0.06) | 0.02 | 0.16 | (0.06) | 0.15 | (0.07) | 0.32 |
| Prostate volume at baseline | 44.20 | (21.40) | 46.76 | (16.13) | 0.03 | 45.29 | (22.54) | 41.72 | (18.38) | 0.10 |
| Number of cores sampled at base biopsy | 8.21 | (2.27) | 8.58 | (2.39) | 0.004 | 8.30 | (2.15) | 8.00 | (2.51) | 0.09 |
| Genetic score based on 33 PCa risk SNPs | 0.94 | (1.83) | 0.77 | (1.81) | 4.95E−09 | 0.93 | (1.84) | 0.96 | (1.80) | 0.66 |

^{†} DRE: Digital rectal examination

TABLE 4
Comparison of characteristics for men in the placebo
group consented or declined genetic studies

| | Consented for genetic studies | | | | |
|---|---|---|---|---|---|
| Variables | Yes | | No | | P-values |
| Number of subjects | 1654 | | 1475 | | |
| Age at baseline | | | | | |
| Mean (SD), years | 62.55 | (6.03) | 62.87 | (6.03) | 0.13 |
| Range | 49-76 | | 49-77 | | |
| # (%) with positive | 214 | (12.94) | 188 | (12.75) | 0.87 |
| family history at baseline | | | | | |
| # (%) with positive | 67 | (4.06) | 51 | (3.47) | 0.39 |
| DRE^{†} at baseline | | | | | |
| Total PSA levels | | | | | |
| at baseline | | | | | |
| Mean (SD), mL | 5.89 | (1.89) | 5.98 | (1.97) | 0.18 |
| Range, mL | 1.8-14.2 | | 2.4-23.2 | | |
| Free/total PSA ratio | | | | | |
| at baseline | | | | | |
| Mean (SD), mL | 0.16 | (0.06) | 0.17 | (0.06) | 0.02 |
| Range, mL | 0.03-0.47 | | 0.04-0.64 | | |
| Prostate volume | | | | | |
| at baseline | | | | | |
| Mean (SD), mL | 46.13 | (17.62) | 44.58 | (17.61) | 0.02 |

| | | |
|---|---|---|
| Range, mL | 3.66-256.83 | 5.75-264.94 |

^{†}DRE: Digital rectal examination

TABLE 5

| | | Random | Random | Random | Random | Random |
|---|---|---|---|---|---|---|
| | | sample | sample | sample | sample | sample |
| | | 1 | 2 | 3 | 4 | 5 |
| FH | 0.53 | | | | | |
| GS33 | 0.59 | | | | | |
| GS15 | | 0.56 | 0.55 | 0.56 | 0.53 | 0.55 |
| GS10 | | 0.54 | 0.54 | 0.54 | 0.56 | 0.53 |

TABLE 6

| | Random | Random | Random | Random | Random |
|---|---|---|---|---|---|
| | Sample 1 | Sample 2 | Sample 3 | Sample 4 | Sample 5 |
| 15 SNPs | rs1465618 | rs1465618 | rs10934853 | rs721048 | rs1465618 |
| | rs12621278 | rs721048 | rs17021918 | rs12621278 | rs721048 |
| | rs7679673 | rs12621278 | rs10486567 | rs2660753 | rs12621278 |
| | rs6465657 | rs17021918 | rs6465657 | rs17021918 | rs10934853 |
| | rs2928679 | rs7679673 | rs2928679 | rs7679673 | rs2928679 |
| | rs1512268 | rs9364554 | rs1512268 | rs9364554 | rs10086908 |
| | rs16901979 | rs6465657 | rs16901979 | rs10486567 | rs620861 |
| | rs620861 | rs10086908 | rs620861 | rs2928679 | rs7127900 |
| | rs10993994 | rs16902094 | rs10993994 | rs10086908 | rs12418451 |
| | rs7127900 | rs620861 | rs7127900 | rs6983267 | rs11649743 |
| | rs12418451 | rs11649743 | rs12418451 | rs4962416 | rs4430796 |
| | rs11649743 | rs1859962 | rs8102476 | rs7127900 | rs1859962 |
| | rs4430796 | rs2735839 | rs887391 | rs12418451 | rs8102476 |
| | rs2735839 | rs9623117 | rs9623117 | rs11649743 | rs9623117 |
| | rs5945619 | rs5759167 | rs5945619 | rs887391 | rs5759167 |
| 10 SNPs | rs17021918 | rs1465618 | rs1465618 | rs1465618 | rs1465618 |
| | rs9364554 | rs12621278 | rs12621278 | rs10934853 | rs12621278 |
| | rs6465657 | rs10934853 | rs1512268 | rs6465657 | rs7679673 |
| | rs2928679 | rs9364554 | rs16901979 | rs2928679 | rs6465657 |
| | rs1512268 | rs10486567 | rs1571801 | rs1512268 | rs1571801 |
| | rs16901979 | rs10086908 | rs10993994 | rs10086908 | rs7127900 |
| | rs1571801 | rs620861 | rs12418451 | rs6983267 | rs8102476 |
| | rs11649743 | rs6983267 | rs11649743 | rs12418451 | rs9623117 |
| | rs1859962 | rs1571801 | rs9623117 | rs11649743 | rs5759167 |
| | rs9623117 | rs7127900 | rs5759167 | rs1859962 | rs5945619 |

TABLE 7
Detection rate of prostate cancer from biopsy in patients with various PSA levels

| | # of patients by PSA levels (ng/mL) | | | | Detection rate of PCa by PSA levels (ng/mL) | | | |
|---|---|---|---|---|---|---|---|---|
| Study | All | <4 | 4-10 | >10 | All | <4 | 4-10 | >10 |
| Goeteborg Round 1 | 740 | 254 | 397 | 89 | 25.9% | 16.5% | 24.9% | 57.3% |
| Goeteborg Rounds 2-6 | 1,241 | 840 | 385 | 16 | 25.9% | 26.5% | 24.7% | 25.0% |
| Rotterdam Round 1 | 2,895 | 769 | 1745 | 381 | 27.6% | 20.2% | 24.8% | 55.9% |
| Rotterdam Rounds 2-3 | 1,494 | 1,019 | 452 | 23 | 26.0% | 23.5% | 31.0% | 39.1% |
| Tarn | 298 | 117 | 161 | 20 | 32.2% | 24.8% | 34.8% | 55.0% |
| SABOR | 392 | 238 | 133 | 21 | 33.9% | 28.2% | 41.4% | 52.4% |
| Cleveland Clinic | 3,286 | 636 | 2059 | 591 | 39.3% | 33.6% | 40.3% | 42.1% |
| Prorect T | 7,324 | 2,967 | 3669 | 688 | 35.1% | 26.1% | 35.6% | 71.4% |
| Tyrol | 5,644 | 2,626 | 2294 | 724 | 27.7% | 20.3% | 30.4% | 45.9% |
| Durham VA | 2,419 | 763 | 1182 | 474 | 47.5% | 39.6% | 43.4% | 70.3% |
| Total | 25,733 | 10,229 | 12477 | 3027 | 33.0% | 25.2% | 33.8% | 56.3% |

PCa: prostate cancer;
PSA: prostate-specific antigen

TABLE 8
Detection rate of prostate cancer in Stockholm-1 and REDUCE study

| Total PSA | # (%) of biopsy patients by GRS | | | | Detection rate (95% CI) of PCa based on GRS | | | |
|---|---|---|---|---|---|---|---|---|
| (ng/mL) | All | <0.5 | 0.5-1.5 | >1.5 | All | <0.5 | 0.5-1.5 | >1.5 |
| 4-6.9 | 2,423 | 283(11.7) | 1,535(63.4) | 605(25) | 43.1(41.1-45.1) | 27.9(22.8-33.5) | 41.3(38.8-43.8) | 54.6(50.5-58.6) |
| 7-9.9 | 1,118 | 118(10.6) | 667(59.7) | 333(29.8) | 47.1(44.2-50.1) | 31.4(23.1-40.5) | 46.8(42.9-50.7) | 53.5(47.9-58.9) |
| >=10 | 958 | 73(7.6) | 583(60.9) | 302(31.5) | 75.1(72.2-77.8) | 67.1(55.1-77.7) | 71.5(67.7-75.2) | 83.8(79.1-87.8) |

PCa: prostate cancer;
PSA: prostate-specific antigen;
GRS: genetic risk score

TABLE 9
Detection rate of prostate cancer in Changhai and Huashan Hospitals, China

| | # (%) of biopsy patients by GRS | | | | Detection rate (95% CI) of PCa based on GRS | | | |
|---|---|---|---|---|---|---|---|---|
| Total PSA | All | <0.5 | 0.5-1.5 | >1.5 | All | <0.5 | 0.5-1.5 | >1.5 |
| 4-9.9 ng/mL | 232 | 28 (12.1) | 143 (61.6) | 61 (26.3) | 17.7(13.0-23.2) | 7.1(0.9-23.5) | 16.8(11.1-23.9) | 24.6(14.5-37.3) |
| 10-19.9 ng/mL | 207 | 26 (12.6) | 139 (67.1) | 42 (20.3) | 35.3(28.8-42.2) | 7.7(1.0-25.1) | 36.7(28.7-44.7) | 47.6(32.0-63.6) |
| >=20 ng/mL | 191 | 20 (10.5) | 105 (55) | 66 (34.6) | 70.7(63.7-77.0) | 55.0(31.5-76.9) | 66.7(56.8-75.6) | 81.8(70.4-90.2) |

PCa: prostate cancer;
PSA: prostate-specific antigen;
GRS: genetic risk score

TABLE 10
SNPs associated with PCa and odds ratio (OR)

| | | | Risk | |
|---|---|---|---|---|
| | | Risk | allele | |
| Chr | SNP | allele | freq | OR |
| 8 | rs16901979 | A | 0.274 | 1.48 |
| 8 | rs1512268 | T | 0.387 | 1.34 |
| 19 | rs6983267 | G | 0.387 | 1.34 |
| 7 | rs1447295 | A | 0.119 | 1.48 |
| 2 | rs103294 | C | 0.241 | 1.34 |
| 13 | rs620861 | G | 0.548 | 1.28 |
| 6 | rs12653946 | T | 0.333 | 1.26 |
| 11 | rs9600079 | T | 0.464 | 1.24 |
| 18 | rs817826 | C | 0.071 | 1.49 |
| 8 | rs339331 | T | 0.655 | 1.23 |
| 10 | rs4430796 | A | 0.75 | 1.2 |
| 8 | rs1465618 | T | 0.643 | 1.17 |
| 8 | rs2252004 | C | 0.732 | 1.17 |

What is claimed is:

1. A method of identifying a subject for whom a prostate biopsy is indicated, comprising:
a) determining, from a nucleic acid sample obtained from the subject, a genotype for the subject at a plurality of biallelic polymorphic loci, wherein each of said plurality has an associated allele and an unassociated allele, wherein the genotype is selected from the group consisting of homozygous for the associated allele, heterozygous, and homozygous for the unassociated allele;
b) calculating a genetic risk score (GRS) for the subject based on the genotype determined in step (a);
c) analyzing the GRS of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated; and
d) performing a prostate biopsy on the subject identified as a subject for whom a prostate biopsy is indicated according to step (c).

2. The method of claim 1, wherein the plurality of biallelic polymorphic loci is a multiplicity, in any combination, of the single nucleotide polymorphisms of Table 1.

3. The method of claim 1, wherein the plurality of biallelic polymorphic loci is the 33 single nucleotide polymorphisms of Table 1.

4. The method of claim 1, wherein the plurality of biallelic polymorphic loci is a multiplicity, in any combination, of the single nucleotide polymorphisms of Table 10.

5. The method of claim 1, wherein the plurality of biallelic polymorphic loci is the 13 single nucleotide polymorphisms of Table 10.

6. The method of claim 1, wherein the subject has a family history of prostate cancer.

7. The method of claim 1, wherein the subject has a prior negative prostate biopsy.

8. A method of determining whether to perform a prostate biopsy on a subject, comprising:
a) determining, from a nucleic acid sample obtained from the subject, a genotype for the subject at a plurality of biallelic polymorphic loci, wherein each of said plurality has an associated allele and an unassociated allele, wherein the genotype is selected from the group consisting of homozygous for the associated allele, heterozygous, and homozygous for the unassociated allele;
b) calculating a genetic risk score (GRS) for the subject based on the genotype determined in step (a);
c) analyzing the GRS of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated;
d) performing a prostate biopsy on the subject if the subject is identified as a subject for whom a prostate biopsy is indicated according to step (c); and
e) not performing a prostate biopsy on the subject if the subject is not identified as a subject for whom a prostate biopsy is indicated according to step (c).

9. A method of identifying a subject for whom a prostate biopsy is indicated, comprising:
a) determining, from a sample obtained from the subject, a p2PSA level, a free PSA (fPSA) level and a total PSA (tPSA) level for the subject;
b) calculating a prostate health index (PHI) for the subject based on the p2PSA level, fPSA level and tPSA level determined in step (a);
c) analyzing the PHI of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated; and
d) performing a prostate biopsy on the subject identified as a subject for whom a prostate biopsy is indicated according to step (c).

10. A method of determining whether to perform a prostate biopsy on a subject, comprising:
a) determining, from a sample obtained from the subject, a p2PSA level, a free PSA (fPSA) level and a total PSA (tPSA) level for the subject;
b) calculating a prostate health index (PHI) for the subject based on the p2PSA level, fPSA level and tPSA level determined in step (a);
c) analyzing the PHI of the subject in combination with a prostate specific antigen (PSA) level of the subject to identify a prostate cancer detection rate for the subject, whereby a prostate cancer detection rate of greater than or equal to a reference value identifies the subject as a subject for whom a prostate biopsy is indicated;
d) performing a prostate biopsy on the subject if the subject is identified as a subject for whom a prostate biopsy is indicated according to step (c); and
e) not performing a prostate biopsy on the subject if the subject is not identified as a subject for whom a prostate biopsy is indicated according to step (c).

11. A chart for determining prostate cancer detection rate as a percentile value, said chart comprising:
a) a first region comprising a first, second and third prostate specific antigen (PSA) value;
b) a second region, adjacent to each respective first, second and third PSA value of the first region, comprising a first, second and third genetic risk score (GRS) value and an average detection rate value (ALL), wherein the first, second and third GRS values are the same for each of the respective PSA values to which the first, second and third GRS values are adjacent;
c) a third region, adjacent to the second region, comprising a reference bar to show a prostate cancer detection rate in percentiles ranging from 1% to 100%; and
d) a fourth region comprising a grid, aligned below the third region and in parallel with the first, second and third GRS values and the ALL value of the second region for each of the respective first, second and third PSA values of the first region, showing a percentile value that specifies a prostate cancer detection rate for each of the first, second and third GRS values and the ALL value in the second region for each of the first, second and third PSA values in the first region.

12. The chart of claim 11, wherein the first PSA value is 4.0-6.9; the second PSA value is 7.0-9.9; and the third PSA value is ≧10.

13. The chart of claim 11, wherein the first PSA value is 4.0-9.9, the second PSA value is 10.0-19.9; and the third PSA value is ≧20.

14. The chart of claim 12, wherein the prostate cancer detection rate is in a range of about 41% to about 47% for a PSA value of 4.0-6.9; the prostate cancer detection rate is in a range of about 22% to about 33% for a PSA value of 4.0-6.9 and a GRS of <0.5; the prostate cancer detection is in a range of about 38% to about 45% for a PSA value of 4.0-6.9 and a GRS of 0.5-1.5; the prostate cancer detection rate is in a range of about 50% to about 59% for a PSA value of 4.0-6.9 and a GRS of >1.5; the prostate cancer detection rate is in a range of about 44% to about 50% for a PSA value of 7.0-9.9; the prostate cancer detection rate is in a range of about 23% to about 41% for a PSA value of 7.0-9.9 and a GRS of <0.5; the prostate cancer detection rate is in a range of about 42% to about 51% for a PSA value of 7.0-9.9 and a GRS of 0.5-1.5; the prostate cancer detection rate is in a range of about 48% to about 59% for a PSA value of 7.0-9.9 and a GRS of >1.5; the prostate cancer detection rate is in a range of about 72% to about 77% for a PSA value of ≧10; the prostate cancer detection rate is in a range of about 55% to about 77% for a PSA value of ≧10 and a GRS of, 0.5; the prostate cancer detection rate is in a range of about 66% to about 75% for a PSA value of ≧10 and a GRS of 0.5-1.5; and a prostate cancer detection rate is in a range of about 78% to about 88% for a PSA value of ≧10 and a GRS of >1.5.

15. The chart of claim 13, wherein the prostate cancer detection rate is in a range of about 13% to about 23% for a PSA value of 4.0-9.9; the prostate cancer detection rate is in a range of about 1% to about 24% for a PSA value of 4.0-9.9 and a GRS of <0.5; the prostate cancer detection is in a range of about 11% to about 24% for a PSA value of 4.0-9.9 and a GRS of 0.5-1.5; the prostate cancer detection rate is in a range of about 14% to about 37% for a PSA value of 4.0-9.9 and a GRS of >1.5; the prostate cancer detection rate is in a range of about 28% to about 42% for a PSA value of 10.0-19.9; the prostate cancer detection rate is in a range of about 1% to about 24% for a PSA value of 10.0-19.9 and a GRS of <0.5; the prostate cancer detection rate is in a range of about 28% to about 45% for a PSA value of 10.0-19.9 and a GRS of 0.5-1.5; the prostate cancer detection rate is in a range of about 31% to about 63% for a PSA value of 10.0-19.9 and a GRS of >1.5; the prostate cancer detection rate is in a range of about 63% to about 75% for a PSA value of ≧20; the prostate cancer detection rate is in a range of about 31% to about 78% for a PSA value of ≧20 and a GRS of <0.5; the prostate cancer detection rate is in a range of about 56% to about 76% for a PSA value of ≧20 and a GRS of 0.5-1.5; and a prostate detection rate is in a range of about 69% to about 90% for a PSA value of ≧20 and a GRS of >1.5.

16. A chart for determining prostate cancer detection rate as a percentile value, said chart comprising:
a) a first region comprising a first, second and third prostate specific antigen (PSA) value;
b) a second region, adjacent to each respective first, second and third PSA value of the first region, comprising a first, second and third prostate health index (PHI) value and an average detection rate value (ALL), wherein the first, second and third PHI values are the same for each of the respective PSA values to which the first, second and third PHI values are adjacent;
c) a third region, adjacent to the second region, comprising a reference bar to show a prostate cancer detection rate in percentiles ranging from 1% to 100%; and
d) a fourth region comprising a grid, aligned below the third region and in parallel with the first, second and third PHI values and the ALL value of the second region for each of the respective first, second and third PSA values of the first region, showing a percentile value that specifies a prostate cancer detection rate for each of the first, second and third PHI values and the ALL value in the second region for each of the first, second and third PSA values in the first region.

17. The chart of claim 16, wherein the prostate cancer detection rate is in a range of about 12% to about 23% for a PSA value of 2.0-9.9; the prostate cancer detection rate is in a range of about 2% to about 13% for a PSA value of 2.0-9.9 and a low (Q1) PHI; the prostate cancer detection is in a range of about 15% to about 30% for a PSA value of 2.0-9.9 and a mid (Q2-Q3) PHI; the prostate cancer detection rate is in a range of about 15% to about 95% for a PSA value of 2.0-9.9 and a high (Q4) PHI; the prostate cancer detection rate is in a range of about 27% to about 43% for a PSA value of 10.0-19.9; the prostate cancer detection rate is in a range of about 1% to about 18% for a PSA value of 10.0-19.9 and a low (Q1) PHI; the prostate cancer detection rate is in a range of about 35% to about 54% for a PSA value of 10.0-19.9 and a mid (Q2-Q3) PHI; the prostate cancer detection rate is in a range of about 35% to about 93% for a PSA value of 10.0-19.9 and a high (Q4) PHI; the prostate cancer detection rate is in a range of about 70% to about 82% for a PSA value of ≧20, independent; the prostate cancer detection rate is in a range of about 1% to about 44% for a PSA value of ≧20 and a low (Q1) PHI; the prostate cancer detection rate is in a range of about 35% to about 58% for a PSA value of ≧20 and a mid (Q2-Q3) PHI; and a prostate detection rate is in a range of about 90% to about 97% for a PSA value of ≧20 and a high (Q4) PHI.
